(19) United States
(12) Patent Application Publication
(10) Pub. No.: US 2024/0083915 A1
(43) Pub. Date: Mar. 14, 2024

(54) PHOTOACTIVE MATERIAL

(71) Applicant: Sumitomo Chemical Co., Ltd., Tokyo (JP)

(72) Inventors: Michal Maciejczyk, Godmanchester (GB); Kiran Kamtekar, Godmanchester (GB); Florence Bourcet, Cambridge (GB)

(73) Assignee: Sumitomo Chemical Co., Ltd., Tokyo (JP)

(21) Appl. No.: 18/268,110

(22) PCT Filed: Dec. 17, 2021
(86) PCT No.: PCT/EP2021/086629

§ 371 Date: Jun. 16, 2023

(30) Foreign Application Priority Data
Dec. 18, 2020 (GB) 2020188.5

(51) Int. Cl. C07D 519/00; H10K 71/15; H10K 85/10; H10K 85/60

(52) U.S. Cl. CPC C07D 519/00; H10K 71/15; H10K 85/111; H10K 85/654; H10K 85/6576; H10K 2101/30

(57) ABSTRACT

A material comprising an electron-accepting unit of formula (I): Ar is an aromatic ring; Ar¹ is a substituted or unsubstituted 5- or 6-membered heteroaromatic ring containing N and C ring atoms; when Ar¹ is a substituted or unsubstituted 6-membered heteroaromatic ring, Ar² is a substituted or unsubstituted 6-membered heteroaromatic ring wherein the ring atoms are selected from N and C; when Ar¹ is a 5-membered heteroaromatic ring, Ar² is a substituted or unsubstituted 5- or 6-membered heteroaromatic ring; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a C atom of Ar³, and where present Ar⁴, with the proviso that at least one X is an electron withdrawing group; and wherein the material further comprises an electron-donating unit.

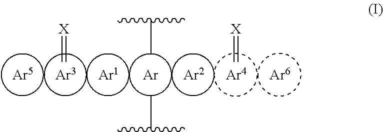

### BACKGROUND

Embodiments of the present disclosure relate to photoactive materials and more specifically, but not by way of limitation, to photoactive materials containing an electron-accepting unit and an electron-donating unit, the materials being suitable for use as an electron-donating material or an electron-accepting material in a photoresponsive device.

Q. Zhang et al, “Novel carbazole-based donor-isoindolo[2,1-a] benzimidazol-11-one acceptor polymers for ternary flash memory and light-emission”, RSC Advances (2019), 9(47), p27665-27673, discloses compounds based on 9-(9-heptadecanyl)-9Hcarbazole and isoindolo[2,1-a]benzimidazol-11-one with fluorine substituents on the acceptor unit.

Q. Zhang et al, “Novel Conjugated Side Chain Fluorinated Polymers Based on Fluorene for Light-Emitting and Ternary Flash Memory Devices”, ChemPubSoc Europe, (2019), 8, p1267-1275, discloses conjugated polymers based on 9,9′-dioctylfluorene and isoindolo[2,1-a]benzimidazol-11-one units with different fluorine substitution.

H. Zhang et al, “Ternary Memory Devices Based on Bipolar Copolymers with Naphthalene Benzimidazole Acceptors and Fluorene/Carbazole Donors”, discloses donor-acceptor-type bipolar conjugated copolymers.

H. Chen et al, “Low Band Gap Donor—Acceptor Conjugated Polymers with Indanone-Condensed Thiadiazolo[3,4-g]quinoxaline Acceptors”, Macromolecules, (2019), 52(16), p6149-6159, discloses compounds of formula (II) and (III) discloses polymers based on 2,5-bis(3-(2-decyltetradecyl)thiophen-2-yl)thieno[3,2-b]-thiophene units as the electron donor and thiadiazolo[3,4-g]quinoxaline acceptor units.

J. Chen et al, “D-A Conjugated Polymers based on Tetracyclic Acceptor Units: Synthesis and Application in Organic Solar Cells”, Macromol. Chem. Phys., (2013), 214(18), p2054-2060, discloses polymers based on indeno-pyrazine and indeno-quinoxaline units.

CN110128631A discloses super low band gap D-A conjugated polymers of formula (I) and (II), for FETs, NIR light detectors, NIR electrochromic devices and biological imaging applications.

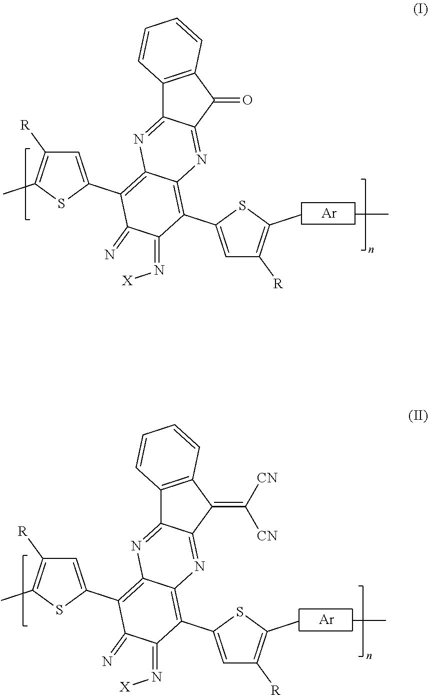

CN101376686A relates to narrow band gap, polymeric donor materials for bulk heterojunction solar cells based on carbazole, fluorene, phenyl and thiophene donor units.

WO2009069717 discloses azaindenofluorenedione derivatives for organic electroluminescent device applications.

### SUMMARY

According to some embodiments, the present disclosure provides a material comprising an electron-accepting unit of formula (I):

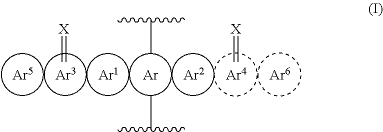

wherein Ar is an aromatic ring; Ar¹ is a substituted or unsubstituted 5- or 6-membered heteroaromatic ring containing N and C ring atoms; when Ar¹ is a substituted or unsubstituted 6-membered heteroaromatic, ring, Ar² is a substituted or unsubstituted 6-membered heteroaromatic ring wherein the ring atoms are selected from N and C; when Ar¹ is a 5-membered heteroaromatic ring, Ar³ is a substituted or unsubstituted 5- or 6 membered heteroaromatic ring; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a C atom of Ar³, and where present, Ar⁴ with the proviso that at least one X is an electron withdrawing group; and wherein the material further comprises an electron-donating unit.

It will be understood that the electron withdrawing group X of Ar³ and Ar⁴ may be attached by its double bond to any available C atom of Ar³ and Ar⁴.

Optionally, the unit of formula (I) is selected from (I-1) to (I-21) according to claim 2.

Optionally, each electron-withdrawing group X is independently selected from O, S and NX⁷⁰ wherein X⁷⁰ is CN, COOR⁸⁰ or C₁ to C₂₀ alkyl chain where any non-terminal C can be replaced by O or S, substituted or unsubstituted 5- or 6-membered aromatic or heteroaromatic ring; and CX¹⁰X¹¹ wherein X¹⁰ and X¹¹ are each independently F, Cl, Br, CN, CF₃ or COOR⁸⁰, wherein R⁸⁰ is H or a substituent.

By “non-terminal” C atom of an alkyl group as used anywhere herein is meant a C atom of the alkyl other than the methyl C atom of a linear (n-alkyl) chain or the methyl C atoms of a branched alkyl chain.

Optionally, each X is an electron-withdrawing group.

In some embodiments, the material is a non-polymeric compound. Optionally the non-polymeric compound is selected from formula (Ia) or (Ib):

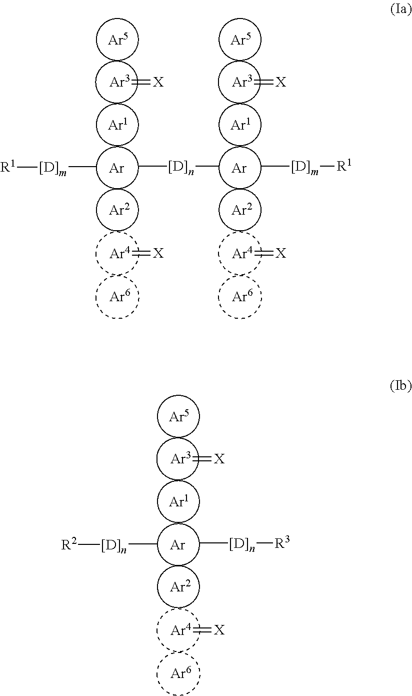

wherein n is at least 1; m is 0, 1, 2 or 3; D in each occurrence is independently an electron-donating unit which may be unsubstituted or substituted with one or more substituents; and R¹ and R² independently in each occurrence is H or a substituent.

In some embodiments, the material is a polymer; the unit of formula (I) is an electron-accepting repeat unit of formula (I); and the electron-donating unit D is an electron-donating repeat unit.

In some embodiments, D of a non-polymeric compound or a repeat unit D of a polymer as described herein is selected from formulae (IIa)—(IIo), according to claim 8.

According to some embodiments, the present disclosure provides a polymer comprising a repeat unit of formula (I):

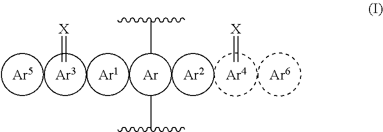

wherein Ar, Ar¹, Ar², Ar³, Ar⁴, Ar⁵, Ar⁶ and X are as described above.

In some embodiments, the repeat unit of formula (I) is selected from (I-1) to (I-21) as described herein.

According to some embodiments, the present disclosure provides a composition comprising an electron donor and an electron acceptor wherein at least one of the electron donor and electron acceptor is a material or polymer according as described herein.

In some embodiments, the electron-acceptor of the composition is the material comprising an electron-accepting unit of formula (I) as described herein. Optionally, the electron acceptor is a non-polymeric compound as described herein.

In some embodiments, the electron donor of the composition is the material comprising an electron-accepting unit of formula (I) as described herein. Optionally, the electron donor is a as described herein.

In some embodiments, the present disclosure provides an organic electronic device comprising an active layer comprising compound or composition as herein described.

Optionally, the organic electronic device is an organic photoresponsive device comprising a bulk heterojunction layer disposed between an anode and a cathode and wherein the bulk heterojunction layer comprises a composition as described herein.

Preferably, the organic photoresponsive device is an organic photodetector.

According to some embodiments, the present disclosure provides a photosensor comprising a light source and an organic photodetector as described herein, wherein the photosensor is configured to detect light emitted from a light source. Optionally, the light source emits light having a peak wavelength of >1100 nm, optionally at least 1200 nm, optionally >1250 nm and more preferably >1300 nm. Optionally, the light source emits light having a peak wavelength of no more than 1600 nm.

According to some embodiments, the present disclosure provides a formulation comprising a material, or composition as described herein, dissolved or dispersed in one or more solvents.

According to some embodiments, the present disclosure provides a method of forming an organic electronic device as described herein, wherein formation of the active layer comprises deposition of a formulation according to claim 24 onto a surface and evaporation of the one or more solvents.

### DESCRIPTION OF DRAWINGS

The disclosed technology and accompanying figures describe some implementations of the disclosed technology.

FIG. 1 illustrates an organic photoresponsive device according to some embodiments.

FIG. 2 shows absorption spectra in toluene solution for a polymer according to an embodiment of the present disclosure and comparative polymers.

FIG. 3 shows current density vs. voltage for an organic photodetector according to some embodiments and comparative organic photodectors, which are not exposed to light;

FIG. 4 shows external quantum efficiencies vs. wavelength for an organic photodetector containing an electron donor polymer according to some embodiments and comparative organic photodetectors containing a comparative electron donor polymer; and

FIG. 5 shows external quantum efficiencies vs. wavelength for organic photodetectors containing an electron donor polymer according to some embodiments of the present disclosure.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

A material as described herein may be provided in a bulk heterojunction layer of a photoresponsive device, preferably a photodetector, in which the bulk heterojunction layer is disposed between an anode and a cathode.

The bulk heterojunction layer comprises an electron donor material and an electron acceptor material wherein at least one of the electron donor material and the electron acceptor material comprises an electron-accepting group of Formula (I):

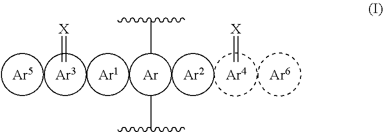

wherein Ar is an aromatic ring; Ar¹ is a substituted or unsubstituted 5- or 6-membered heteroaromatic ring containing N and C ring atoms; when Ar¹ is a substituted or unsubstituted 6-membered heteroaromatic ring, Ar² is a substituted or unsubstituted 6-membered heteroaromatic ring wherein the ring atoms are selected from N and C; when Ar¹ is a 5-membered heteroaromatic ring, Ar² is a substituted or unsubstituted 5- or 6 membered heteroaromatic ring; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a C atom of Ar³, and where present, Ar⁴ with the proviso that at least one X is an electron withdrawing group; and wherein the material further comprises an electron-donating unit.

It will be understood that the electron withdrawing group X of Ar³ and Ar⁴ may be attached by its double bond to any available C atom of Ar³ and Ar⁴.

Carbon atoms of Ar¹, Ar², Ar³, Ar⁴, Ar⁵ and Ar⁶ which are not fused to another ring or substituted with X carry a group R⁶¹ wherein R⁶¹ in each occurrence is independently H or a substituent. Substituents R⁶¹ are preferably selected from the group consisting of:
F;
Cl;
CN;
NO₂;
   linear, branched or cyclic C₁₋₂₀ alkyl wherein one or more non-adjacent, non-terminal C atoms may be replaced by O, S, NR⁷, CO or COO wherein R⁷, is a C₁₋₁₂ hydrocarbyl and one or more H atoms of the C₁₋₂₀ alkyl may be replaced with F; and
   a group of formula (Ak)u-(Ar⁷)v wherein Ak is a C₁₋₁₂ alkylene chain in which one or more non-adjacent C atoms may be replaced with O, S, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₁₂ alkyl wherein one or more non-adjacent C atoms may be replaced with O, S, CO or COO Preferably, Ar⁷ is phenyl.

More preferred substituents R⁶¹ are F; Cl; C₁₋₂₀ alkyl wherein one or more H atoms may be replaced with F; and phenyl which is unsubstituted or substituted with one or more substituents selected from F and C₁₋₁₂ alkyl wherein one or more H atoms of the alkyl may be replaced with F.

It will be understood that the possibility of substituting Ar¹, Ar², Ar⁵ and Ar⁶ will be dependent on the structure of formula (I) and the availability of substitution positions. For example, if Ar² is present and is a 6-membered aromatic or heteroaromatic ring containing less than four heteroatoms in the ring, then substitution may be present; if Ar² is a 5-membered heteroaromatic ring, containing less than three heteroatoms in the ring, then substitution may be present; if Ar⁵ is a monocyclic or polycyclic group containing a least one aromatic ring, then substitution may be present.

Preferably, the unit of formula (I) is selected from (I-1) to (I-21):

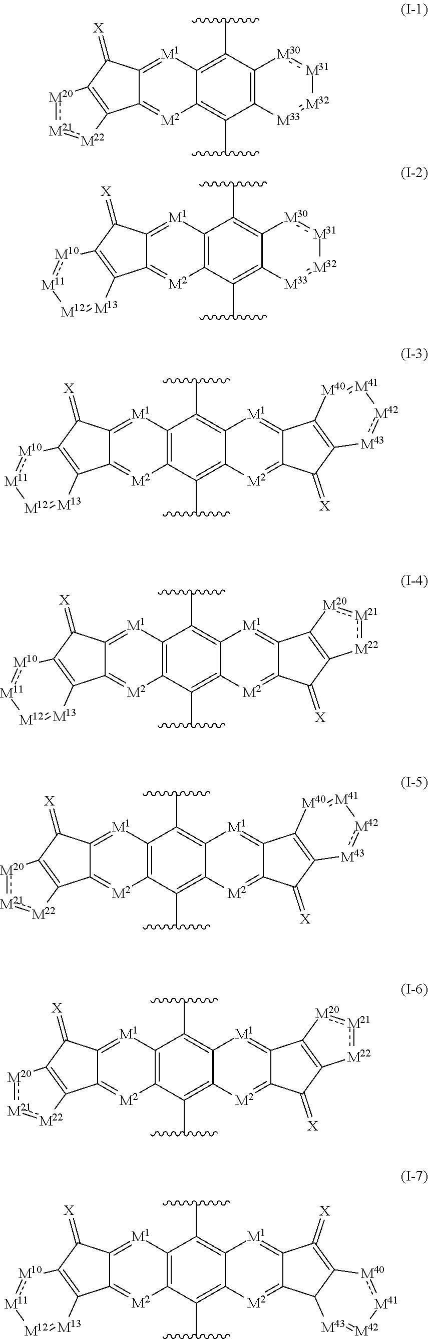

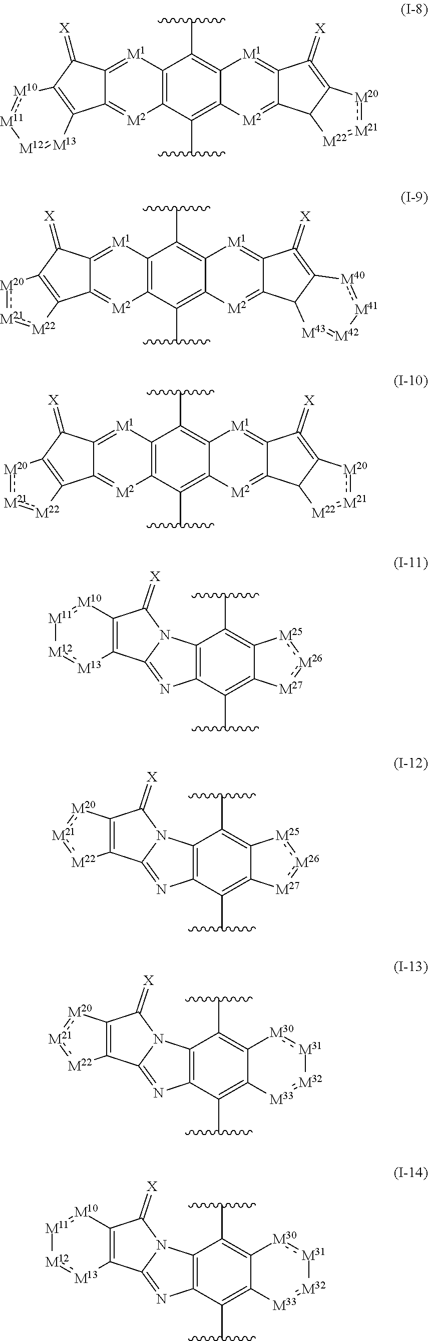

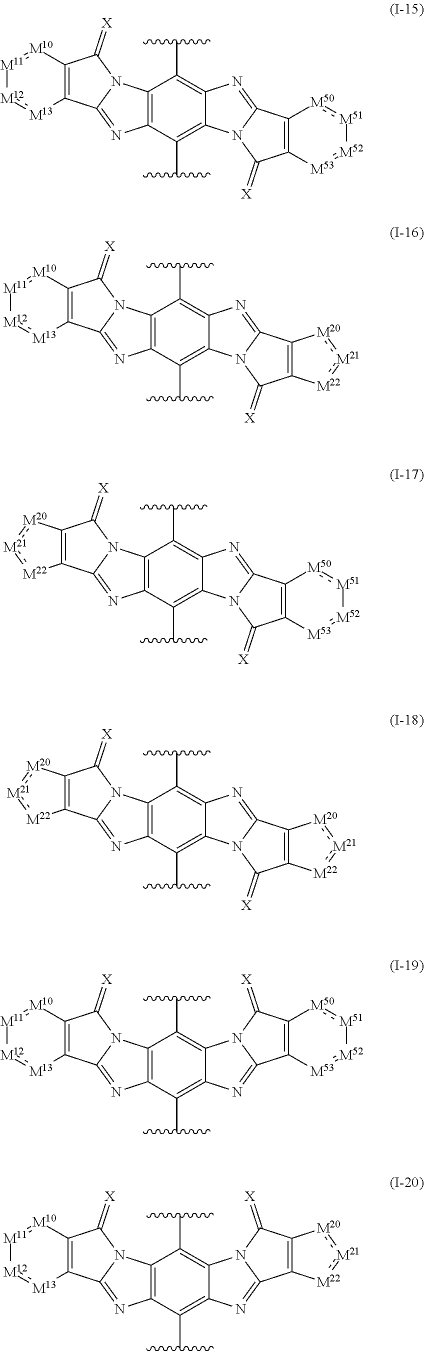

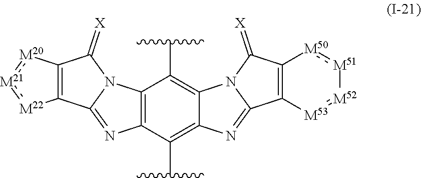

wherein
M¹ and M² is independently CR⁶¹ or N wherein R⁶¹ in each occurrence is H or a substituent, preferably H or a substituent as described above;
M¹⁰, M¹¹, m¹², m¹³, m²⁰, m²¹, m²², m⁴⁰, m⁴¹, m⁴², m⁴³, m⁵⁰, m⁵¹, m⁵², and m⁵³ is independently N, S, O or CR⁶¹, wherein R⁶¹ in each occurrence is H or a substituent and with the proviso that a S or O is not adjacent to another S or O;
M³⁰, m³¹, M³² and M³³ is independently N, or CR⁶¹;
M²⁵, M²⁶ and M²⁷ is independently N, S, O or CR⁶¹; with the proviso that a N or O is not adjacent to another N or O; and
X is independently an electron withdrawing group.

Preferably, Ar is benzene.

Preferably, Ar¹ is a substituted or unsubstituted 5- or 6-membered heteroaromatic ring consisting of N and C ring atoms; consisting of N, C and O ring atoms; or consisting of N, C and S ring atoms.

Preferably no more than 2 ring atoms of Ar¹ are N atoms.

Optionally, no more than 1 ring atom of Ar¹ is an O or S atom.

Preferably, Ar¹ is selected from imidazole, pyridine, thiazine, pyrazine, and oxazine.

More preferably, Ar¹ is selected from imidazole and pyrazine.

When Ar¹ is a substituted or unsubstituted 6-membered heteroaromatic ring, Ar² is a substituted or unsubstituted 6-membered heteroaromatic ring wherein the ring atoms are selected from N and C.

Preferably no more than 2 ring atoms of Ar² are N atoms.

Preferably, Ar² is selected from pyridine, pyrazine, thiazine and oxazine.

More preferably, Ar² is pyrazine.

Preferably, when Ar¹ is a 5-membered heteroaromatic ring, Ar² is a substituted or unsubstituted 5- or 6-membered heteroaromatic ring consisting of N and C ring atoms; consisting of N, C and O rings atoms; or consisting of N, C and S ring atoms.

Optionally, no more than 1 ring atom of Ar² is an O or S atom.

Preferably, Ar² is selected from pyrrole, pyrazole, imidazole, oxazole, thiazole, pyridine, thiazine, pyrimidine, pyridazine, pyrazine, thiadiazole, oxadiazole, oxazine, and triazole.

Preferably, Ar² is selected from, thiadiazole, triazole, pyrimidine, pyridazine and pyrazine.

More preferably, Ar² is thiadiazole, triazole and pyrazine.

Preferably Ar³ is a 5-membered carbocyclic ring.

Preferably, Ar⁴ where present is a 5-membered carbocyclic ring.

Preferably, Ar⁵ is independently a substituted or unsubstituted monocyclic or polycyclic group containing at least one aromatic or heteroaromatic ring, wherein the heteroaromatic ring consists of N and C ring atoms; consists of N, C and O ring atoms; or consists of N, C and S ring atoms.

Preferably, Ar⁵ is independently a substituted or unsubstituted monocyclic or polycyclic group containing at least one aromatic or heteroaromatic ring, wherein the heteroaromatic ring consists of N and C ring atoms and optionally O and S ring atoms.

Preferably no more than 2 ring atoms of Ar⁵ are N atoms.

Optionally, no more than 1 ring atom of Ar⁵ is an O or S atom.

Preferably, Ar⁵ is selected from benzene, pyrrole, pyrazole, imidazole, oxazole, thiazole, pyridine, thiazine, a diazine including pyrimidine, pyridazine, pyrazine, thiadiazole, oxadiazole, oxazine, and triazole.

Preferably, Ar⁵ is selected from benzene, thiadiazole, triazole and a diazine for example pyrimidine, pyridazine or pyrazine.

More preferably, Ar⁵ is benzene.

Preferably, Ar⁶ where present is selected from groups as defined for Ar⁵.

Preferably, each electron-withdrawing group X is independently selected from O, S and NX⁷⁰ wherein X⁷⁰ is CN or COOR⁸⁰; and CX¹⁰X¹¹ wherein X¹⁰ and X¹¹ are each independently F, Cl, Br, CN, CF₃, NO₂, or COOR⁸⁰, wherein R⁸⁰is H or a substituent and preferably a C₁₋₂₀ hydrocarbyl group, and preferably each of X¹⁰ and X¹¹ is F.

Optionally, each electron-withdrawing group is NX⁷⁰, wherein X⁷⁰ is selected from C₁₋₂₀alkyl wherein one or more non-adjacent, non-terminal C atoms may be replaced with O, S, COO or CO and one or more H atoms of the alkyl may be replaced with F; phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F; and a heteroaromatic group which is unsubstituted or substituted with one or more substituents.

Preferably, each electron-withdrawing group X is independently selected from O and NX⁷⁰ wherein X⁷⁰ is CN or COOR⁸⁰; and CX¹⁰X¹¹ wherein X¹⁰ and X¹¹ are each independently selected from CN or CF₃.

Preferably, each electron-withdrawing group X is independently selected from O and CX¹⁰X¹¹ wherein X¹⁰ and X¹¹ are each independently CN or COOR⁸⁰.

More preferably, each electron-withdrawing group X is independently selected from O and CX¹⁰X¹¹ wherein X¹⁰ and X¹¹ are each CN.

In a preferred embodiment, Ar is benzene; Ar¹ is a 5- or 6-membered heteroaromatic ring containing N and C atoms; Ar² is a substituted or unsubstituted 6-membered heteroaromatic ring wherein the ring atoms are selected from N and C; or Ar² is a substituted or unsubstituted 5- or 6-membered heteroaromatic ring, when Ar¹ is a 5-membered ring; Ar³ is a 5-membered ring or a substituted or unsubstituted 6-membered ring; Ar⁴ is a 5-membered ring or substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently an electron withdrawing group, bound to the C atoms of Ar³ and/or Ar⁴; and wherein the material further comprises conjugated electron-donating unit D.

In a more preferred embodiment, Ar is benzene; Ar¹ is a 6-membered heteroaromatic ring containing N and C atoms; Ar² is a substituted 6-membered heteroaromatic ring; Ar³ is a 5-membered ring; Ar⁵ is monocyclic containing one aromatic ring; and X is an electron withdrawing group bound to the C atom of Ar³; and wherein the material further comprises a conjugated electron-donating unit D.

In a preferred embodiment, Ar is benzene; Ar¹ is a 6-membered heteroaromatic ring containing N and C atoms; Ar² is a substituted or unsubstituted 6-membered heteroaromatic ring wherein the ring atoms are selected from N and C; Ar³ is a 5-membered ring; Ar⁴ where present is a 5-membered ring; Ar⁵ is a substituted or unsubstituted monocyclic or polycyclic group containing at least one aromatic or heteroaromatic ring; Ar⁶ where present is as substituted or unsubstituted monocyclic or polycyclic group containing at least one aromatic or heteroaromatic ring; and each X is independently an electron withdrawing group bound to the C atoms of Ar³ and/or Ar⁴; and wherein the material further comprises a conjugated electron-donating unit D.

In a preferred embodiment, Ar is benzene; Ar¹ is a 5-membered heteroaromatic ring containing N and C atoms; Ar² is a substituted or unsubstituted 6-membered heteroaromatic ring; Ar³ is a 5-membered ring or a substituted or unsubstituted 6-membered ring; Ar⁴ is a 5-membered ring or substituted or unsubstituted 6-membered ring or is absent; Ar⁵ is independently a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently an electron withdrawing group bound to the C atoms of Ar³ and/or Ar⁴; and wherein the material further comprises a conjugated electron-donating unit.

In a preferred embodiment, Ar is benzene; Ar¹ is a 5-membered heteroaromatic ring containing N and C atoms; Ar³ is a substituted or unsubstituted 5-membered heteroaromatic ring; Ar³ is a 5-membered ring or a substituted or unsubstituted 6-membered ring; Ar⁴ is a 5-membered ring or substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently an electron withdrawing group bound to the C atoms of Ar³ and/or Ar⁴; and wherein the material further comprises a conjugated electron-donating unit D.

Exemplary units of formula (I) are illustrated below which is unsubstituted or substituted with one or more substituents R⁶¹ as described above:

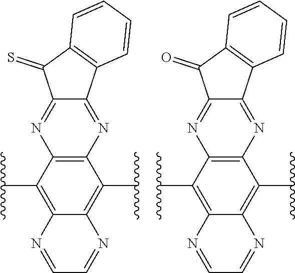

In some embodiments, the material comprising the unit of formula (I) has an absorption peak greater than 750 nm.

In some embodiments, the material comprising the unit of formula (I) has an absorption peak greater than 900 nm.

In some embodiments, the material comprising the unit of formula (I) has an absorption peak greater than 1100 nm.

In some embodiments the material comprising the unit of formula (I) has an absorption peak in the range of 750-2000 nm, between 750-1400 nm, between 750-900 nm or 900-2000 nm.

Unless stated otherwise, absorption spectra of materials as described herein are measured using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution.

Absorption data are obtained by measuring the intensity of transmitted radiation through a solution sample. Absorption intensity is plotted vs. incident wavelength to generate an absorption spectrum. A method for measuring film absorption, may comprise measuring a 15 mg/ml solution in a quartz cuvette and comparing to a cuvette containing the solvent only.

Unless stated otherwise, absorption data as provided herein is as measured in toluene solution.

The electron donor (p-type) material has a HOMO deeper (further from vacuum) than a LUMO of the electron acceptor (n-type) material. Optionally, the gap between the HOMO level of the p-type donor material and the LUMO level of the n-type acceptor material is less than 1.4 eV. Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO =4.8-E ferrocene (peak to peak average)−E reduction of sample (peak maximum).

HOMO =4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

In some embodiments, the bulk heterojunction layer contains only one electron donor material and only one electron acceptor material, at least one of the donor and acceptor comprising an electron-accepting unit of formula (I).

In some embodiments, the bulk heterojunction layer contains two or more electron donor materials and/or two or more electron acceptor materials.

In some embodiments, the weight of the donor material(s) to the acceptor material(s) is from about 1:0.5 to about 1:2, preferably about 1:1.1 to about 1:2.

In some embodiments, the material comprising the group of formula (I) is a non-polymeric compound containing at least one unit of formula (I), optionally 1, 2 or 3 units of formula (I) and at least on electron-donating unit D. Preferably, the non-polymeric compound has a molecular weight of less than 5,000 Daltons, optionally less than 3,000 Daltons. Preferably, the non-polymeric compound contains no more than 3 groups of formula (I).

In some embodiments, the material comprising the group of formula (I) is a polymer comprising a repeat unit of formula (I) and an electron-donating repeat unit, more preferably alternating electron-accepting repeat units of formula (I) and electron-donating repeat units.

Preferably, the polystyrene-equivalent number-average molecular weight (Mn) measured by gel permeation chromatography of the polymer is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

In some embodiments the polymer may be part of a composition comprising or consisting of an electron-accepting (n-type) material and an electron-donating (p-type) material wherein the polymer is the electron-donating material. The composition may comprise one or more further materials, e.g. one or more further electron-donating materials and/or one or more further electron-accepting materials.

Preferably, each unit of formula (I) is bound directly to at least one electron-donating unit D.

A non-polymeric compound comprising a unit of formula (I) may have formula (Ia) or (Id):

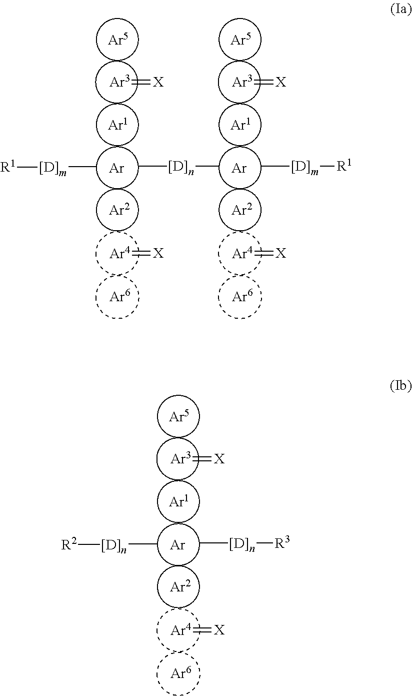

wherein n is at least 1, optionally 1, 2 or 3; m is 0, 1, 2 or 3; D in each occurrence is independently an electron-donating unit which may be unsubstituted or substituted with one or more substituents; and R¹ and R² independently in each occurrence is H or a substituent.

Optionally, R¹ and R² are each independently selected from the group consisting of H; an electron withdrawing group including but not limited to F, CN, and NO₂; C₁₋₂₀ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

In some embodiments the polymer comprising repeat units of formula (I) may contain the repeating structure of formula (Ic), comprising the repeat unit of formula (I) and an adjacent conjugated electron-donating repeat unit D:

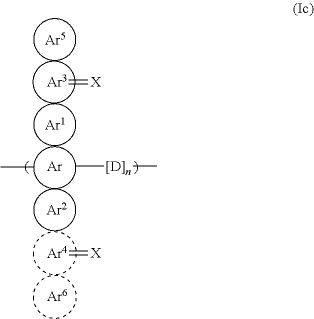

wherein n is at least 1, optionally 1, 2 or 3.

For an electron donor material or electron acceptor material containing an electron-accepting unit of formula (I) and an electron-donating unit D the, or each, unit of formula (I) has a LUMO level that is deeper (i.e. further from vacuum) than the, or each, electron-donating unit, preferably at least 1 eV deeper. The LUMO levels of an electron-donating unit and an electron-accepting unit of formula (I) may be as determined by modelling, respectively, the LUMO level of D-H or H-D-H and H-[Formula (I)]-H, respectively, i.e. by replacing the bond or bonds between D and formula (I) with a bond or bonds to a hydrogen atom.

Preferably, a model compound of formula H-[Formula (I)]-H containing one or more electron-withdrawing groups deepens the LUMO by at least 0.2 eV as compared to the case where the electron-withdrawing groups are absent.

### Electron-Donating Unit

Electron-donating units D are preferably in each occurrence a monocyclic or polycyclic heteroaromatic group which contains at least one furan or thiophene and which may be unsubstituted or substituted with one or more substituents. Preferred electron-donating units D are monocyclic thiophene or furan or a polycyclic donor wherein each ring of the polycyclic donor includes thiophene or furan rings and, optionally, one or more of benzene, cyclopentane, or a six-membered ring containing 5 C atoms and one of N and O atoms.

Optionally, electron donating units D are selected from formulae (IIa)-(IIq), or a combination thereof:

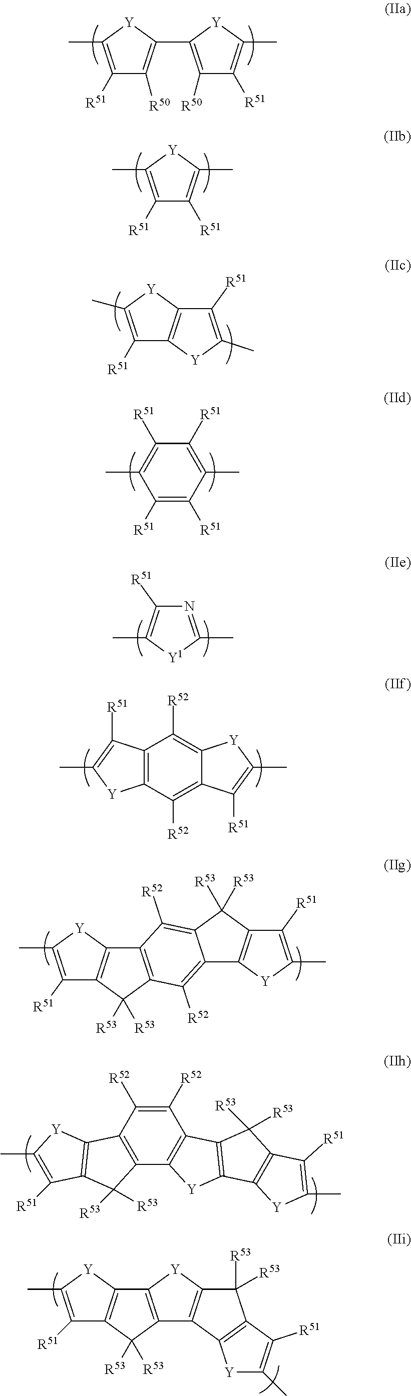

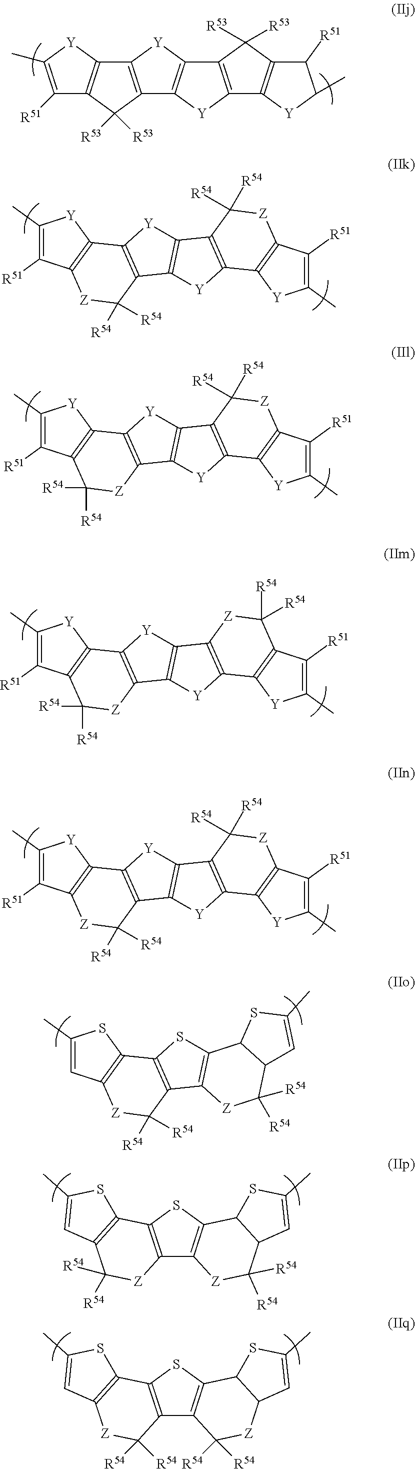

wherein Y and Y¹ in each occurrence is independently O, S or NR⁵⁵, Z in each occurrence is O, S, NR⁵⁵ or C(R⁵⁴)₂; R⁵⁰, R⁵¹, R⁵² R⁵⁴ and R⁵⁵ independently in each occurrence is H or a substituent wherein R⁵⁰ groups may be linked to form a ring; and R⁵³ independently in each occurrence is a substituent.

In some embodiments, the electron-donating unit D in each occurrence in the material of formula (I) is a single unit of formula (IIa)-(IIq) connected directly to at least one electron-accepting unit, e.g. an electron-donating repeat unit connected directly to adjacent electron-accepting repeat units.

In some embodiments, the material of formula (I) comprises a plurality of directly linked electron-donating units D, preferably electron-donating units selected from formulae (IIa)-(IIq). Each electron-donating unit D in a chain of two or more D units may be the same or the chain may contain two or more different D units. The individual units in a chain of D units may be linked in any orientation.

Preferably, the material comprising the group of formula (I) comprises an electron-donating group D of formula (IIa-1):

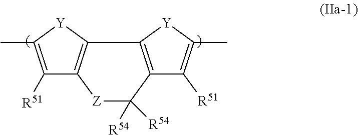

Wherein Y, Z, R⁵¹ and R⁵⁴ are as described above.

Y of formula (IIa) is preferably S.

Z of formula (IIa) is preferably O or S.

In some preferred embodiments, the only electron-donating unit or units D of the material comprising the group of formula (I) are units of formula (IIa-1). According to these embodiments, each unit of formula (IIa-1) is preferably directly linked to at least one electron-accepting unit of formula (IIa-1).

In some preferred embodiments, the material comprising the group of formula (I) comprises two or more different electron-donating units, preferably two or more different electron donating units selected from formulae (IIa)-(IIq). According to these embodiments, the material comprises:
an electron-donating unit selected from formulae (IIa-1) and (IIf)-(IIq), preferably formula (IIa-1) and
an electron-donating unit selected from formulae (IIb)-(IIe), preferably (IIb) or (IIc), disposed between the electron-accepting unit of formula (I) and the electron-donating unit selected from formulae (IIa-1) and (IIf)-(IIq).

Preferably, the electron-donating unit selected from formulae (IIb)-(IIe) is directly linked to the electron-accepting unit of formula (I) and to the electron-donating unit selected from formulae (IIa-1) and (IIf)-(IIq), thereby providing a bridge unit between the electron-accepting unit of formula (I) and the electron-donating unit selected from formulae (IIa-1) and (IIf)-(IIq).

A chain of directly linked electron-donating units D may be linked in any orientation. For example, in the case where each electron-donating unit D is a group of formula (IIa-1) and n is 2, −[D]ₙ-may be selected from any of:

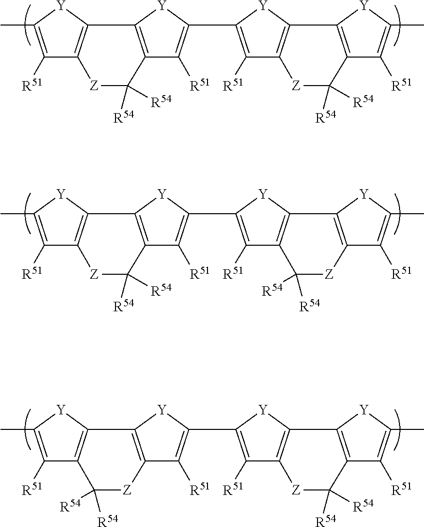

Optionally, R⁵⁰, R⁵¹ and R⁵² independently in each occurrence are selected from H; F; C₁₋₂₀ alkyl wherein one or more non-adjacent, non-terminal C atoms may be replaced with O, S, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g. phenyl.

The one or more substituents of Ar³, if present, may be selected from C₁₋₁₂ alkyl wherein one or more non-adjacent, non-terminal C atoms may be replaced with O, S, COO or CO and one or more H atoms of the alkyl may be replaced with F.

Preferably, each R⁵⁴ is selected from the group consisting of:
H;
F;
   linear, branched or cyclic C₁₋₂₀ alkyl wherein one or more non-adjacent, non-terminal C atoms may be replaced by O, S, NR⁷, CO or COO wherein R⁷, is a C₁₋₁₂ hydrocarbyl and one or more H atoms of the C₁₋₂₀ alkyl may be replaced with F; and a group of formula (Ak)u-(Ar⁷)v wherein Ak is a C₁₋₁₂ alkylene chain in which one or more non-adjacent C atoms may be replaced with O, S, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₁₂ alkyl wherein one or more non-adjacent C atoms may be replaced with O, S, CO or COO

Preferably, Ar⁷ is phenyl.

Preferably, each R⁵¹ is H.

Optionally, R⁵³ independently in each occurrence is selected from C₁₋₂₀ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

Preferably, R⁵⁵ is H or C₁₋₃₀ hydrocarbyl group Preferably, each R⁵⁰ is a substituent. Exemplary repeat units of formula (IIa-1) include, without limitation:

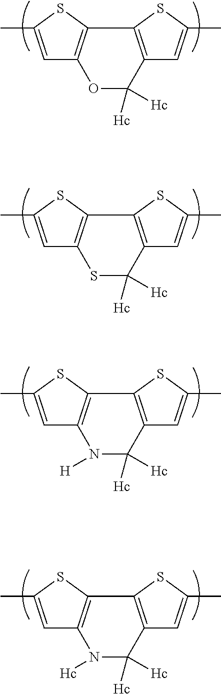

wherein Hc in each occurrence is independently a C₁₋₂₀ hydrocarbyl group, e.g. C₁₋₂₀ alkyl, unsubstituted aryl, or aryl substituted with one or more C₁₋₁₂ alkyl groups. The aryl group is preferably phenyl.

### Polymer Synthesis and Monomers

A polymer as described herein may be formed by polymerising a monomer for forming electron-donating repeat unit D and a monomer for forming the electron-accepting repeat unit of formula (I). The polymerisation method includes, without limitation, methods for forming a carbon-carbon bond between an aromatic carbon atom of an electron-donating unit D and an aromatic carbon atom of an electron-accepting unit (I).

In some embodiments, formation of the polymer comprises polymerisation of a monomer of formula (Xa) and a monomer of formula (Xb):

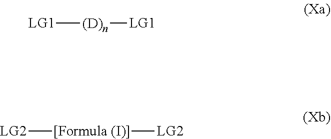

In some embodiments, formation of the polymer comprises polymerisation of a monomer of formula (Xc) and a monomer of formula (Xd):

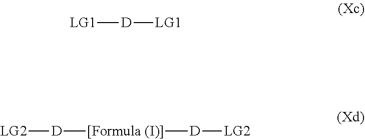

LG1 is a first leaving group bound to an aromatic carbon atom.

LG2 is a second leaving group bound to an aromatic carbon atom which is different from LG1.

A carbon-carbon bond is formed during polymerisation between aromatic carbon atoms to which LG1 and LG2 are bound.

It will be understood that a repeat unit as described anywhere herein may be formed from a monomer comprising or consisting of the repeat unit and leaving groups. For example, polymerisation of Formula (Xd) forms a repeat unit including D and Formula (I) repeat units.

Optionally, LG1 is selected from one of group (a) and group (b), and LG2 is selected from the other of group (a) and group (b):
(a) halogen or —OSO₂R⁸ wherein R⁸ is an optionally substituted C₁₋₁₂ alkyl group or optionally substituted aryl group;
(b) boronic acid and esters thereof; and —SnR⁹₃ wherein R⁹ independently in each occurrence is a C₁₋₁₂ hydrocarbyl group.

Suitable polymerisation methods include, without limitation, Suzuki polymerisation and Stille polymerisation. Suzuki polymerisation is described in, for example, WO 00/53656.

In some embodiments, each LG1 may be one of: (i) a halogen or —OSO₂R⁶; or (ii), a boronic acid or ester, and each LG2 may be the other of (i) and (ii).

In some embodiments, each LG1 may be one of: (i) a halogen or —OSO₂R⁶; and (iii) —SnR⁹₃, and each LG2 may be the other of (i) and (iii).

Optionally, R⁶ in each occurrence is independently a C₁₋₁₂ alkyl group which is unsubstituted or substituted with one or more F atoms; or phenyl which is unsubstituted or substituted with one or more F atoms.

—OSO₂R⁶ is preferably tosylate or triflate.

Exemplary boronic esters have formula (VIII):

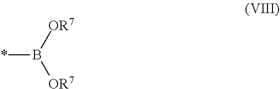

wherein R⁷, in each occurrence is independently a C₁₋₂₀ alkyl group, * represents the point of attachment of the boronic ester to an aromatic ring of the monomer, and the two groups R⁷, may be linked to form a ring which is unsubstituted or substituted with one or more substituents, e.g. one or more C₁₋₆ alkyl groups.

Optionally, R⁷, independently in each occurrence is selected from the group consisting of C₁₋₁₂ alkyl; unsubstituted phenyl; and phenyl substituted with one or more C₁₋₆ alkyl groups.

In a preferred embodiment, the two groups R⁷ are linked, e.g. to form:

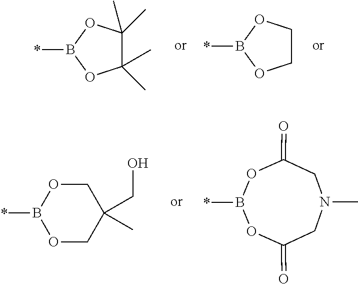

A halogen leaving group is preferably Br or I.

### Electron Donor Material

In the case where the material comprising the group of formula (I) is an electron-accepting material, it may be used with any electron donor material containing a group of formula (I) or any other electron donor material known to the person skilled in the art, including organic polymers and non-polymeric organic molecules.

In a preferred embodiment the electron donor material is an organic conjugated polymer, which can be a homopolymer or copolymer including alternating, random or block copolymers. Preferred are non-crystalline or semi-crystalline conjugated organic polymers. Further preferably the p-type organic semiconductor is a conjugated organic polymer with a narrow band gap, typically between 2.5 eV and 1.5 eV, preferably between 2.3 eV and 1.8 eV.

Optionally, the p-type donor has a HOMO level no more than 5.5 eV from vacuum level. Optionally, the p-type donor has a HOMO level at least 4.1 eV from vacuum level.

As exemplary p-type donor polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3 -substituted thiophene), poly(3,4-bisubstituted thiophene), polyselenophene, poly(3 -substituted selenophene), poly(3,4- bisubstituted selenophene), poly(bisthiophene), poly(terthiophene), poly(bisselenophene), poly(terselenophene), polythieno[2,3-b]thiophene, polythieno [3,2-b]thiophene, polybenzothiophene, polybenzo [1,2-b :4,5 -b′j dithiophene, polyisothianaphthene, poly(mono sub stituted pyrrole), poly(3,4-bisubstituted pyrrole), poly-1,3,4-oxadiazoles, polyisothianaphthene, derivatives and co-polymers thereof may be mentioned. Preferred examples of p-type donors are copolymers of polyfluorenes and polythiophenes, each of which may be substituted, and polymers comprising benzothiadiazole-based and thiophene-based repeating units, each of which may be substituted. It is understood that the p-type donor may also consist of a mixture of a plurality of electron donating materials.

Exemplary electron donor polymers comprising a repeat unit of formula (I) include polymers having a repeating structure selected from:

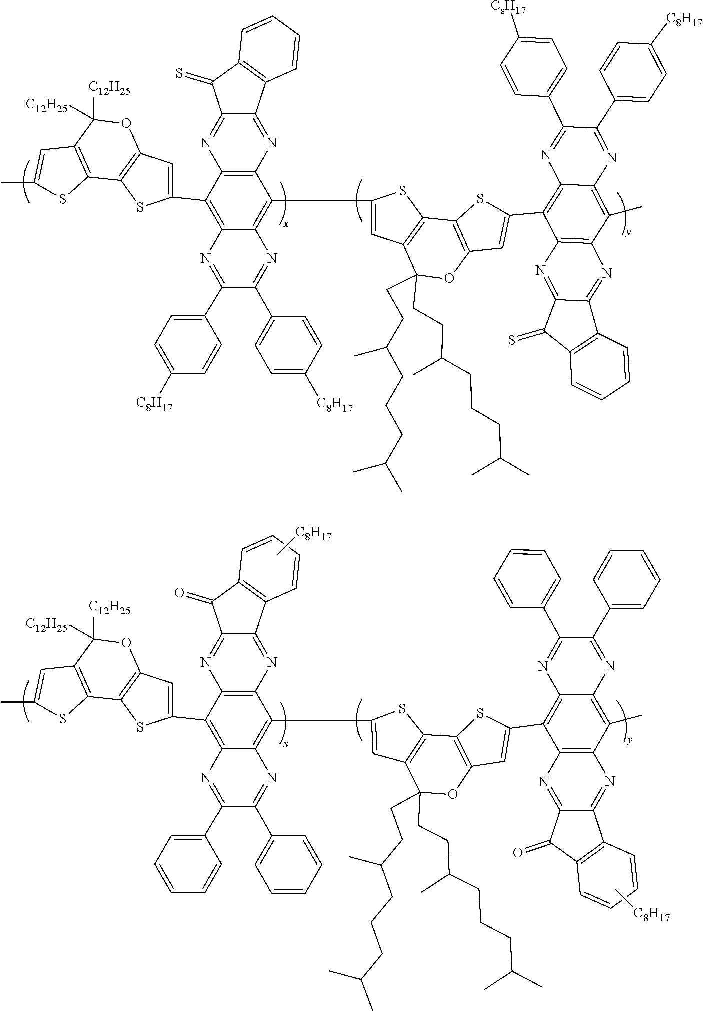

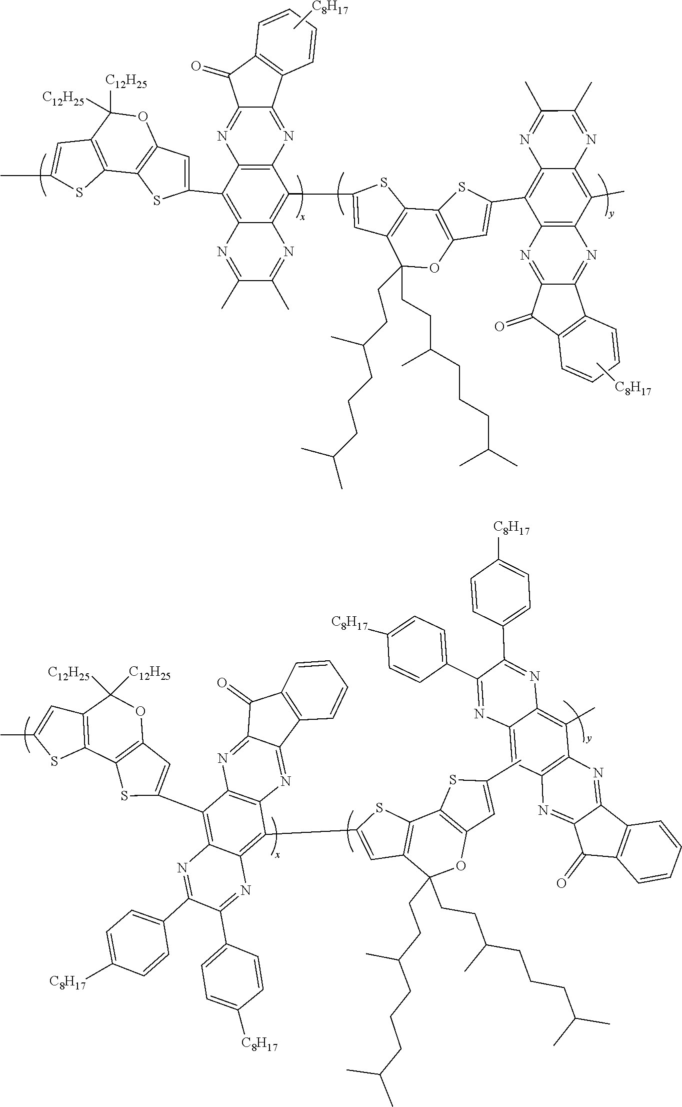

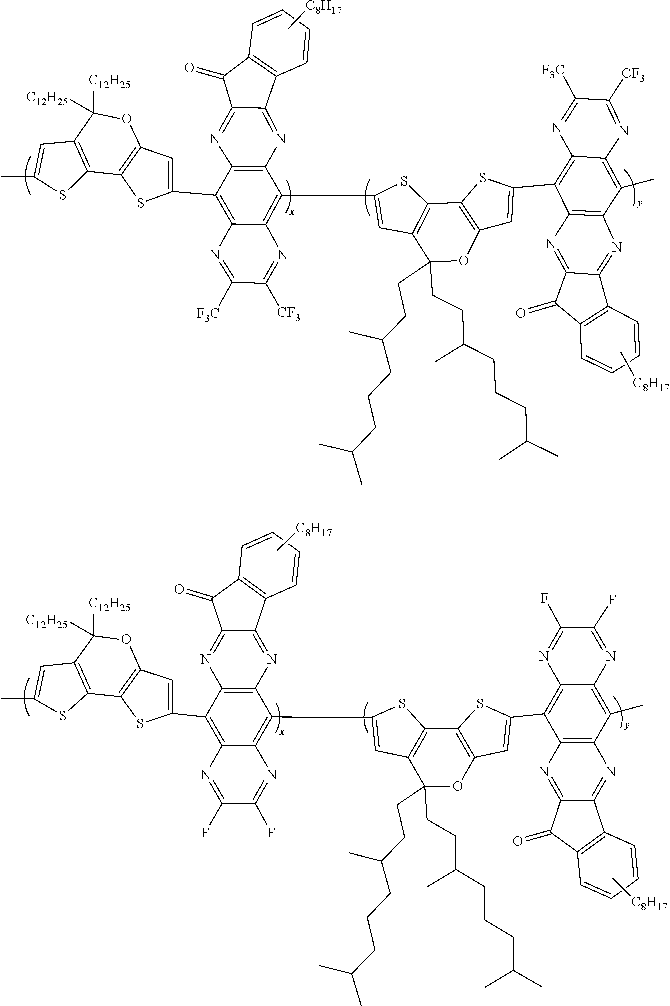

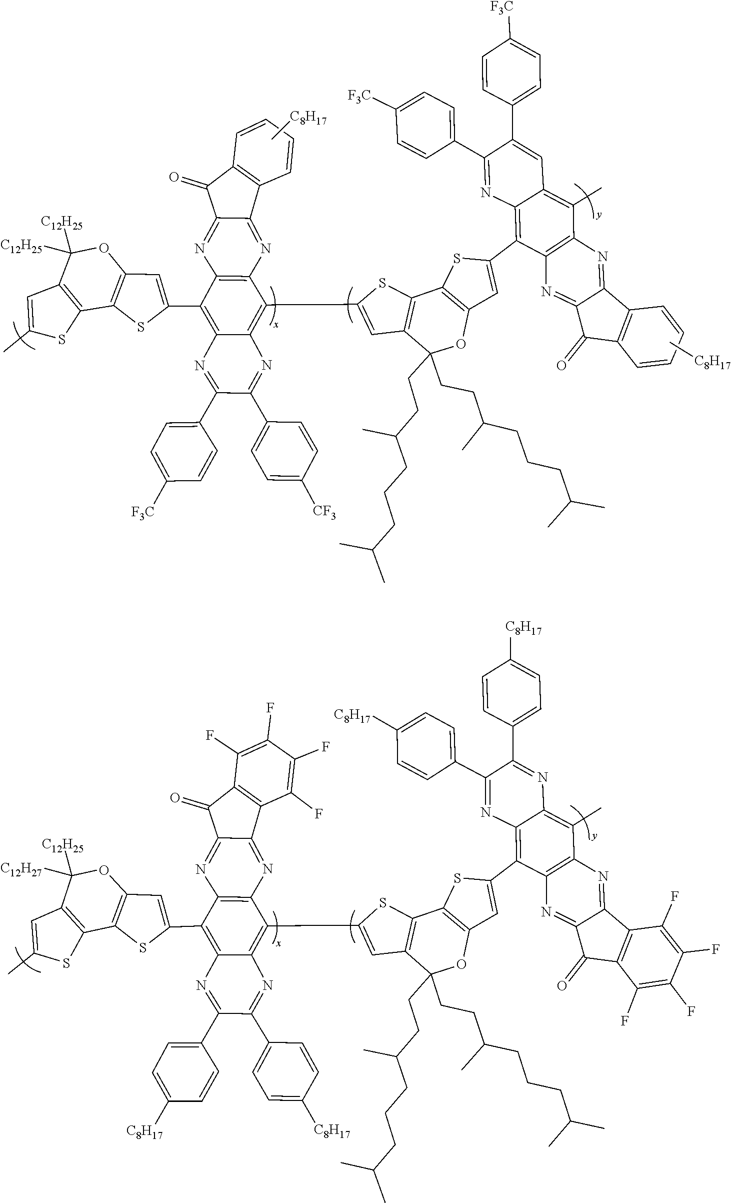

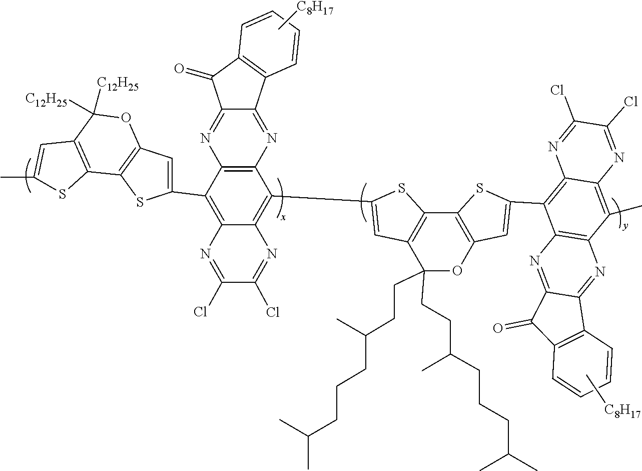

wherein x and y are each an amount of the illustrated repeating structures; x/(x+y)=1; 0≤x≤1; 0≤y≤1; and x+y=1.

Optionally, in the case where the electron donor polymer does not contain a repeat unit of formula (I), it comprises a repeat unit selected from repeat units of formulae:

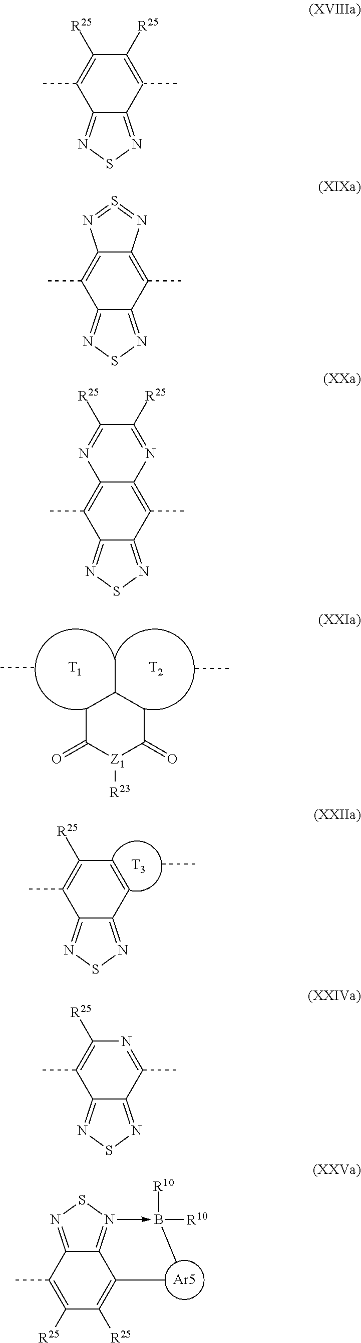

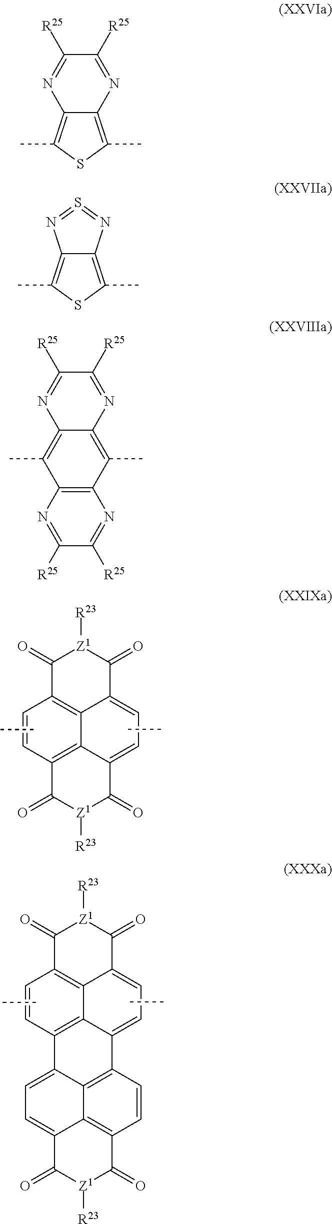

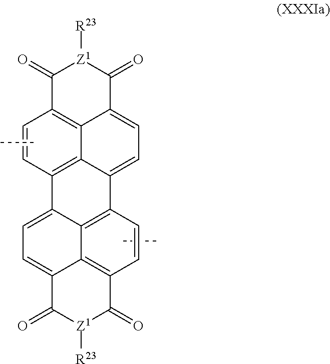

R²³ in each occurrence is a substituent, optionally C₁₋₁₂ alkyl wherein one or more non-adjacent, non-terminal C atoms may be replaced with O, S, COO or CO and one or more H atoms of the alkyl may be replaced with F.

R²⁵ in each occurrence is independently H; F; CN; NO₂; C₁₋₁₂alkyl wherein one or more non-adjacent, non-terminal C atoms may be replaced with O, S, COO or CO and one or more H atoms of the alkyl may be replaced with F; an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO; or

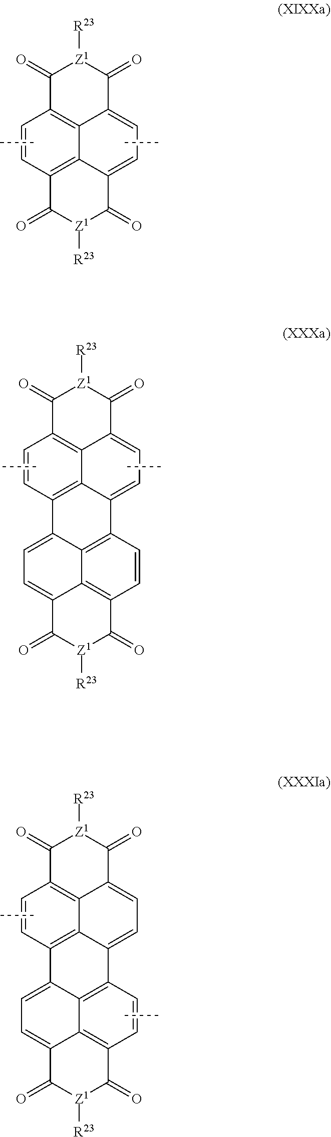

wherein Z⁴⁰, z⁴¹, Z⁴² and Z⁴³ are each independently CR¹³ or N wherein R¹³ in each occurrence is H or a substituent, preferably a C₁₋₂₀ hydrocarbyl group;

Y⁴⁰ and Y⁴¹ are each independently O, S, NX⁷¹ wherein X⁷¹ is CN or COOR⁴⁰; or CX⁶⁰x⁶¹ wherein X⁶⁰ and X⁶¹ is independently CN, CF₃ or COOR⁴⁰;

W⁴⁰ and W⁴¹ are each independently O, S, NX⁷¹ wherein X⁷¹ is CN or COOR⁴⁰; or CX⁶⁰X⁶¹ wherein X⁶⁰ and ⁶¹ is independently CN, CF₃ or COOR⁴⁰; and

R⁴⁰ in each occurrence is H or a substituent, preferably H or a C₁₋₂₀ hydrocarbyl group.

Z¹ is N or P.

T¹, T² and T³ each independently represent an aryl or a heteroaryl ring, optionally benzene, which may be fused to one or more further rings. Substituents of T¹, T² and T³, where present, are optionally selected from non-H groups of R²⁵.

R¹⁰ in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Exemplary donor materials are disclosed in, for example, WO2013051676, the contents of which are incorporated herein by reference.

### Electron Acceptor Material

In the case where the material comprising the group of formula (I) is an electron donor material, it may be used with any electron-accepting material containing a group of formula (I) or any other electron-accepting material known to the person skilled in the art.

Exemplary electron-accepting materials are non-fullerene acceptors, which may or may not contain a unit of formula (I), and fullerenes. A composition containing the material comprising the group of formula (I) may comprise only one electron-accepting material or it may comprise two or more electron-accepting materials, for example at least one non-fullerene acceptor and at least one fullerene acceptor.

Exemplary electron-accepting compounds containing at least one unit of formula (I) include:

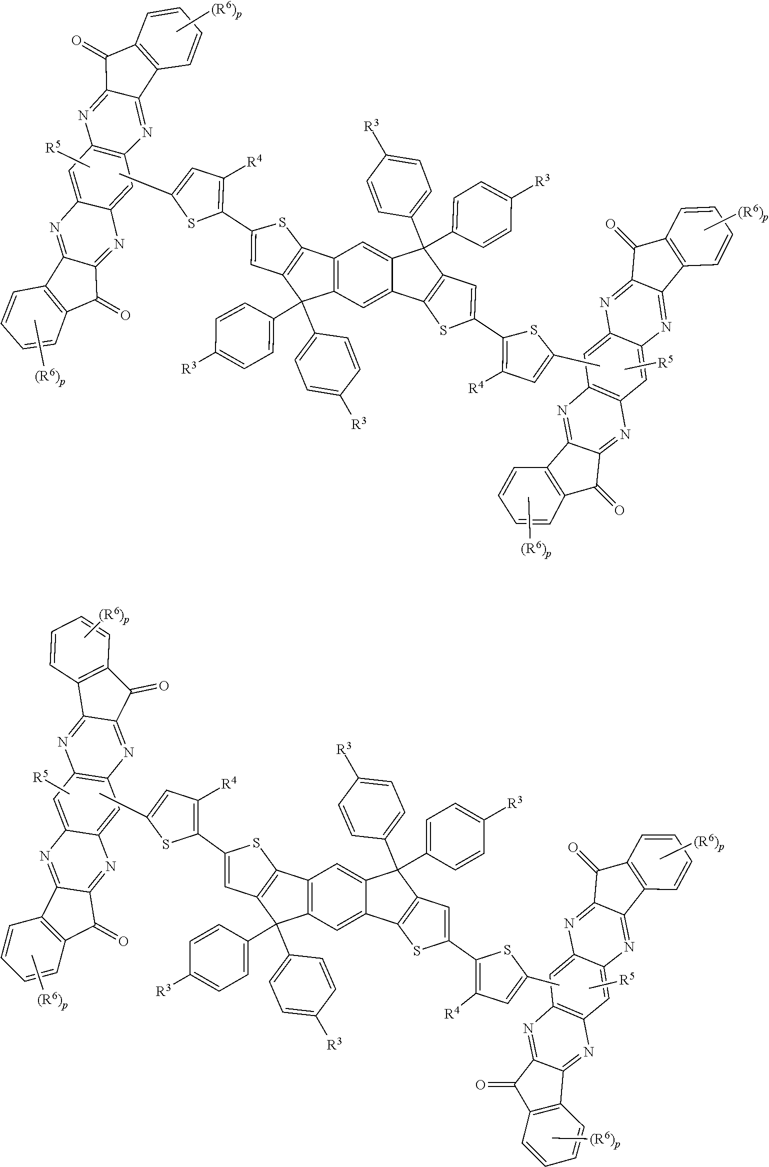

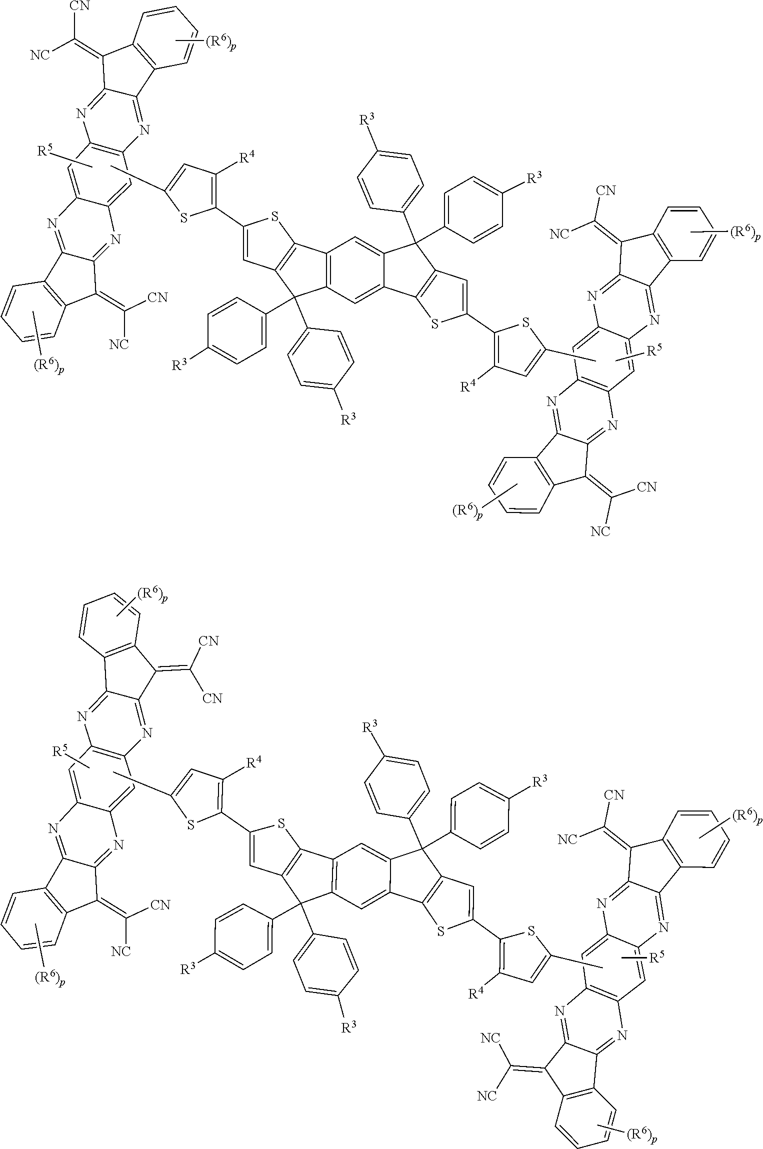

wherein:

R³ and R⁴ in each occurrence is independently a C₁₋₁₂ alkyl group wherein one or more non-adjacent, non-terminal C atoms may be replaced with O, S, CO or COO;

R⁵ in each occurrence is independently R⁶¹ as described above, preferably H or a C₁₋₁₂ alkyl wherein one or more non-adjacent, non-terminal C atoms may be replaced with O, S, CO or COO; or an aryl group, preferably phenyl, which may be unsubstituted or substituted with one or more substituents, preferably one or more C₁₋₁₂ alkyl or alkoxy substituents

R⁶ in each occurrence is independently selected from substituents described with reference to R⁶¹, preferably F; Cl; a C₁₋₁₂ alkyl wherein one or more non-adjacent, non-terminal C atoms may be replaced with O, S, CO or COO and one or more H atoms may be replaced with F; and an aryl group, preferably phenyl, which may be unsubstituted or substituted with one or more substituents, preferably one or more substituents selected from F and C₁₋₁₂ alkyl or alkoxy substituents wherein one or more H atoms may be replaced with F.

p in each occurrence is independently 0, 1, 2, 3, or 4.

Non-fullerene acceptors which do not contain a unit of formula (I) are described in, for example, Cheng et. al., “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F.

Exemplary fullerene electron acceptor materials are C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (C₆₀ThCBM); and optionally substituted fullerenes in which a C═C bond is replaced with two C═O bonds and/or a C atom is replaced with N, for example as in KLOC-6.

Fullerene derivatives may have formula (IV):

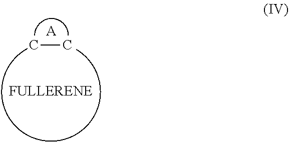

wherein A, together with the C—C group of the fullerene, forms a monocyclic or fused ring group which may be unsubstituted or substituted with one or more substituents.

Exemplary fullerene derivatives include formulae (IVa), (IVb) and (IVc):

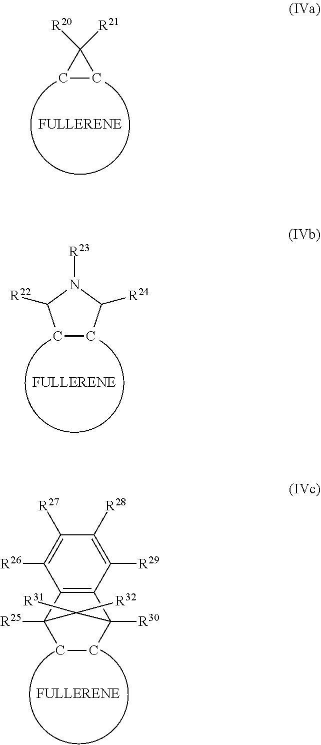

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent, non-terminal C atoms may be replaced with O, S, CO or COO and one or more H atoms may be replaced with F.

Substituents of aryl or heteroaryl, where present, are optionally selected from C₁₋₁₂ alkyl wherein one or more non-adjacent, non-terminal C atoms may be replaced with O, S, CO or COO and one or more H atoms may be replaced with F.

### Formulations

The bulk heterojunction layer may be formed by any process including, without limitation, thermal evaporation and solution deposition methods.

Preferably, the bulk heterojunction layer is formed by depositing a formulation comprising the electron donor material(s), the electron acceptor material(s) and any other components of the bulk heterojunction layer dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

### Organic Electronic Device

A polymer or composition as described herein may be provided as an active layer of an organic electronic device. In a preferred embodiment, a bulk heterojunction layer of an organic photoresponsive device, more preferably an organic photodetector, comprises a composition as described herein.

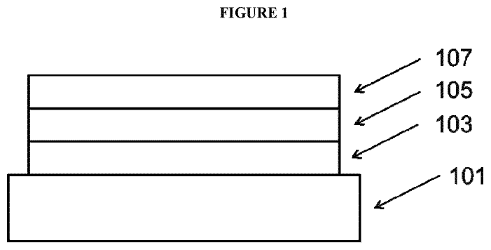

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent.

Each transparent electrode preferably has a transmittance of at least 70%, optionally at least 80%, to wavelengths in the range of 750-1000 nm or 1300-1400 nm. The transmittance may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron acceptor material. The bulk heterojunction layer may consist of these materials or may comprise one or more further materials, for example one or more further electron donor materials and/or one or more further electron acceptor materials.

### Applications

A circuit may comprise the OPD connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm, optionally in the range of 900-1000 nm. In some embodiments, the light source has a peak wavelength greater than 1000 nm, optionally in the range of 1300-1400 nm.

The present inventors have found that a material comprising an electron-accepting unit of formula (I) may be used for the detection of light at longer wavelengths, particularly >1300-1400 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

Intermediate Compound Example 1

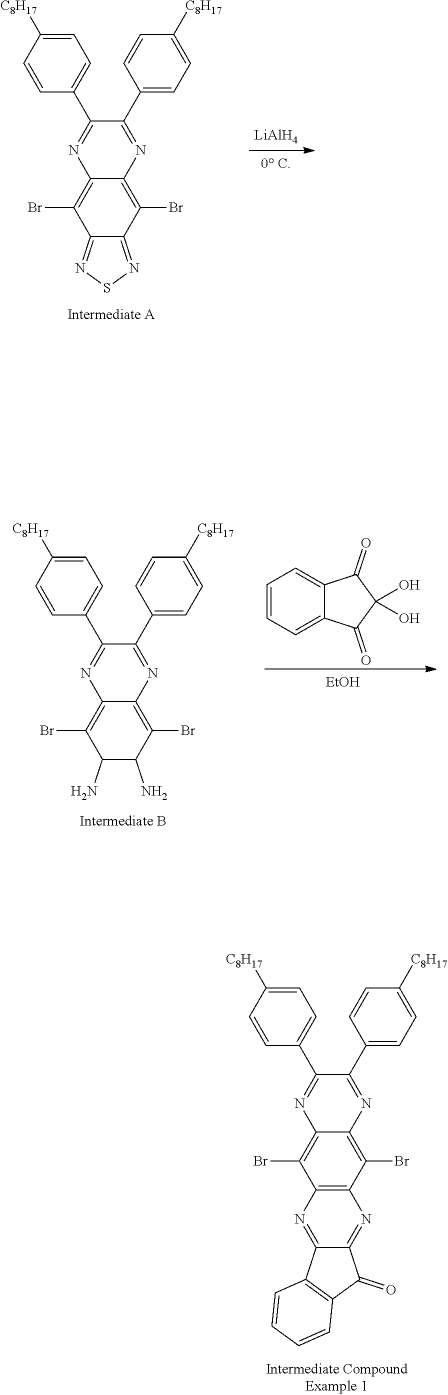

A solution of Intermediate A (6.85 g, 9.48 mmol, prepared as described in Bioconjugate Chemistry, 2016, 27(7), p1614-1623) in THF (257 ml) was cooled to 0° C. LiAlH₄ (37.92 ml, 37.92 mmol, 1M in THF) was added dropwise. After 30 minutes the reaction mixture was quenched with water, evaporated dissolved in ethyl acetate and filtered. Precipitation from heptane, gave Intermediate B (4.24 g) as a yellow solid.

A solution of Intermediate B (4.24 g, 5.87 mmol) and ninhydrin (4.18 g, 23.47 mmol) in ethanol (20 ml) was heated at reflux overnight. The reaction mixture was cooled, and the resulting orange precipitate was filtered, washed with ethanol and recrystallized (isopropanol/chloroform) to give Intermediate Compound Example 1 (3.70 g).

¹H NMR (400 MHz, CDCl₃), δ [ppm]: 8.38 (d, 1H, 7.6Hz); 8.05 (d, 1H, 7.8 Hz); 7.88 (t, 1H, 7.5Hz); 7.69-7.74 (m, 5H); 7.20-7.22 (m, 4H); 2.64-2.67 (m, 4H); 1.61-1.65 (m, 4H); 1.27-1.32 (m, 20H); 0.88 (t, 6H, 7.1Hz).

### Intermediate Compound Example 2

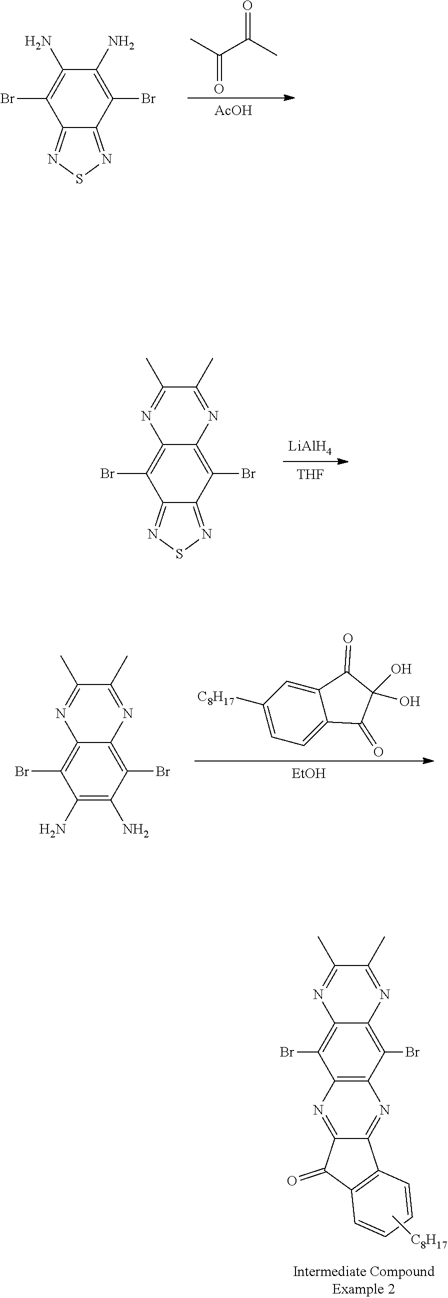

Intermediate Compound Example 2 may be prepared in a similar manner as described for Intermediate Compound Example 1.

Intermediate Compound Example 1 or Intermediate Compound Example 2 may be reacted to form polymeric or non-polymeric materials comprising an electron-accepting unit derived from these compounds and an electron-donating unit.

Polymer Example 1-2 may be formed by Suzuki-Miyara polymerisation of Intermediate Compound Example 1-2 with a monomer for forming an electron-donating repeat unit, for example as disclosed in U.S. Pat. No. 9,512,149, the contents of which are incorporated herein by reference.

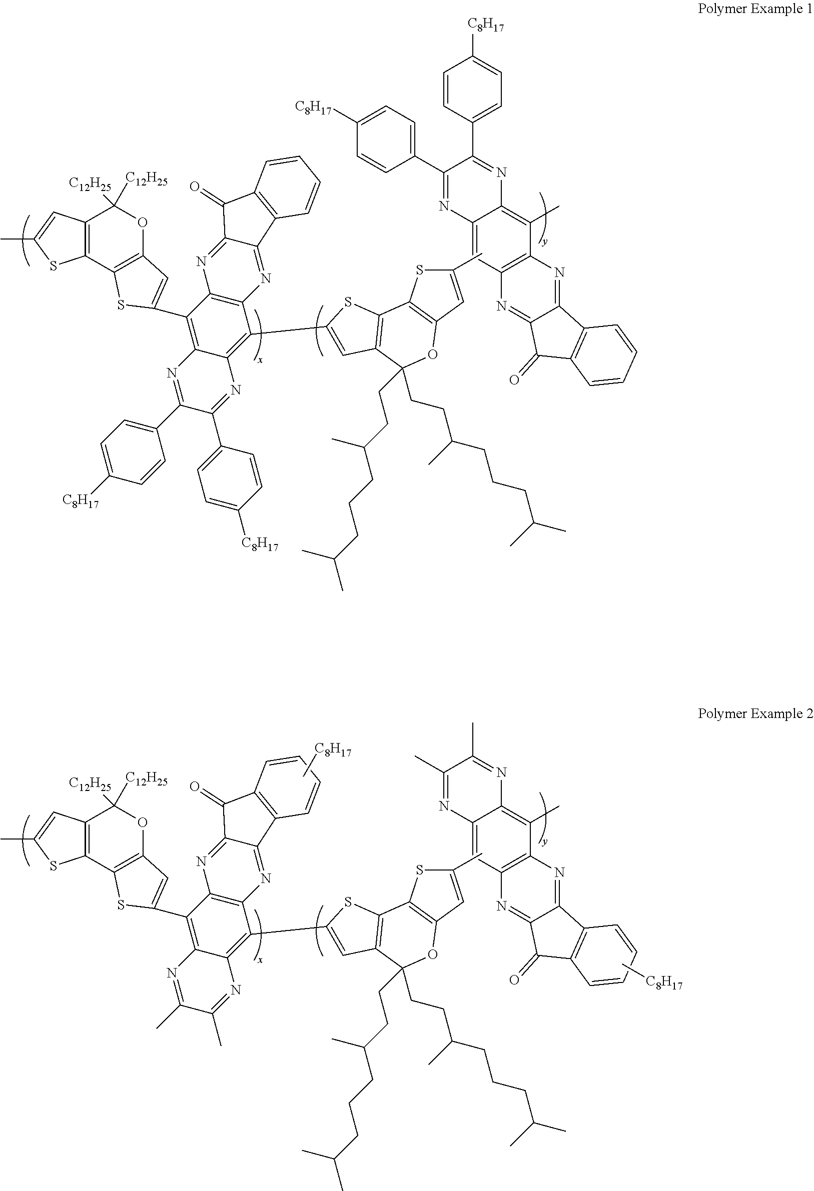

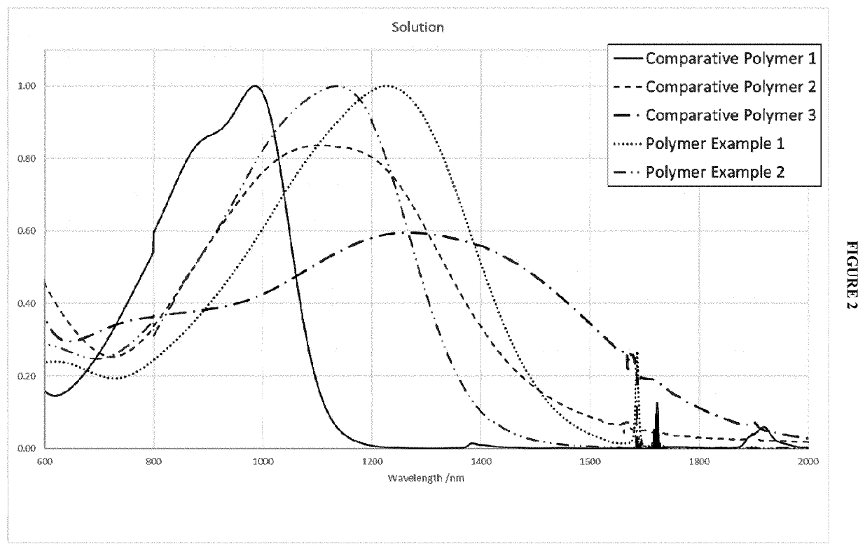

With reference to the absorption spectra of FIG. 2, recorded in toluene solution, Polymer Example 1 shows stronger absorption in the range of about 1100-1400 nm than any of Comparative Polymers 1-3, illustrated below, and Polymer Example 2 shows stronger absorption than any of the comparative polymers at wavelengths above about 1300 nm.

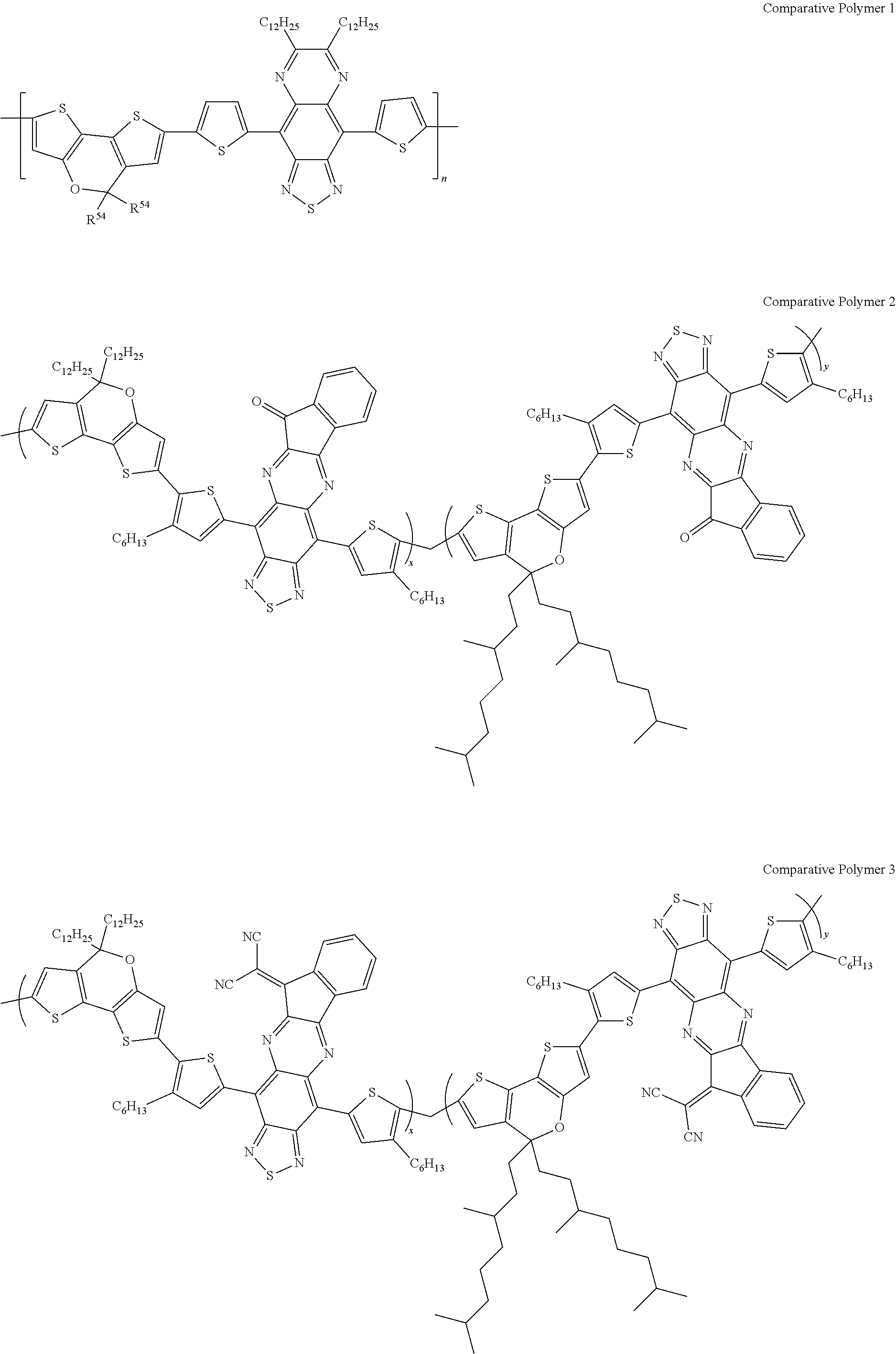

Table A contains HOMO and LUMO values as measured by SWV for Polymer Examples 1 to 4 and Comparative Polymer 1.

| Polymer | HOMO (eV) | LUMO (eV) | Band gap (eV) |
|---|---|---|---|
| Comparative Polymer 1 | −5.05 | −3.45 | 1.6 |
| Comparative Polymer 2 | −5.07 | −3.77 | 1.3 |
| Comparative Polymer 3 | −5.10 | −3.95 | 1.15 |
| Polymer Example 1 | −4.84 | −3.74 | 1.1 |
| Polymer Example 2 | −4.88 | −3.63 | 1.25 |

Intermediate Compound Example 3

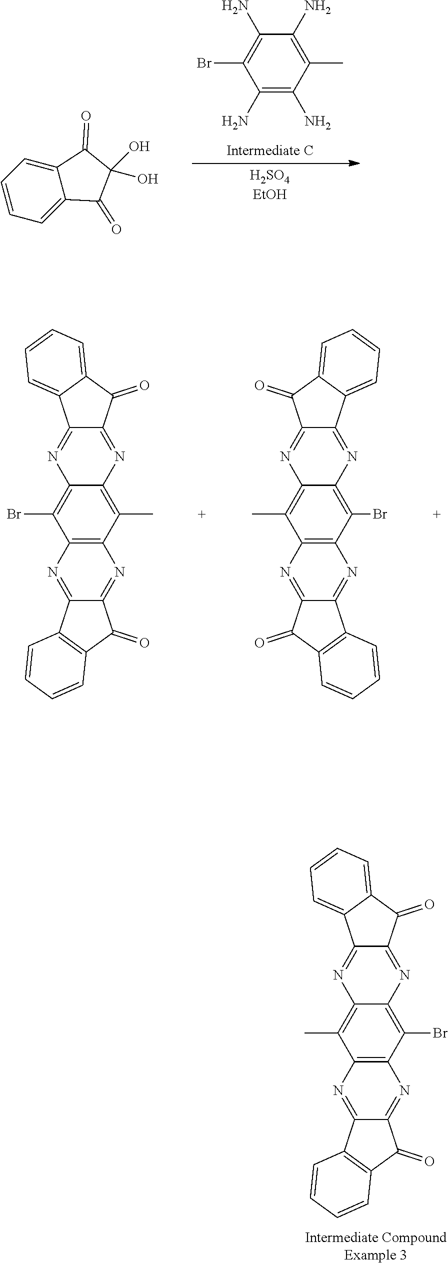 [SMILES: O=C1c2ccccc2C(=O)C1(O)O ; Cc1c(N)c(N)c(Br)c(N)c1N ; Cc1c2nc3c(=O)c4ccccc4c3nc2c(Br)c2nc3c(nc12)c(=O)c1ccccc13 ; Cc1c2nc3c(=O)c4ccccc4c3nc2c(Br)c2nc3c(nc12)c(=O)c1ccccc13 ; "H2SO4EtOH" ; Cc1c2nc3c(nc2c(Br)c2nc4c(=O)c5ccccc5c4nc12)c(=O)c1ccccc13]

Intermediate Compound Example 3 may be prepared in a similar manner as described for Intermediate Compound Example 1. Intermediate C may be synthesized as disclosed in US20190051781 via the method reported in Org. Lett., Vol. 13, No. 1, 2011.

A non-polymeric material comprising an electron-accepting repeat unit formed by reaction of Intermediate Compound Example 2 may be formed, for example as shown below.

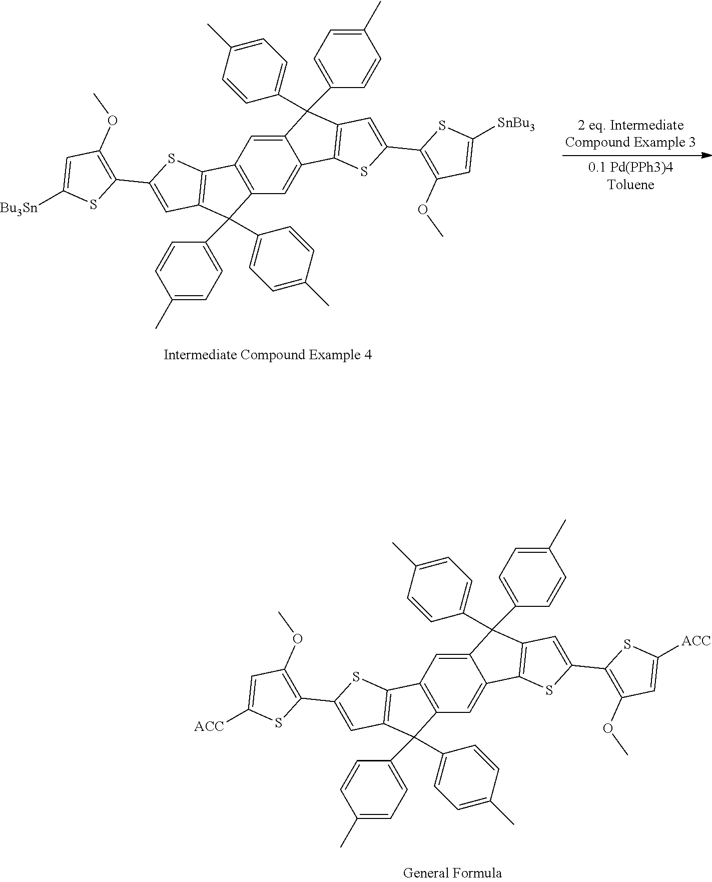

Intermediate Compound Example 4 may be prepared via standard lithiation and stannylation methods, analogous to that disclosed in for example US20190181348.

### General Formula A

Intermediate Compound Example 4 (1 equiv.), Intermediate Compound Example 3 (2.2 equiv.) and Pd(PPh₃)₄ (0.1 equiv.) are dissolved in toluene under nitrogen and heated to 100° C. for 48 hours. The mixture is allowed to cool, poured into dilute aqueous KF, extracted with DCM and purified. This is analogous to that disclosed in for example CN104557968.

### Modelling Example 1

All modelling as described in these examples was performed using Gaussian09 software available from Gaussian using Gaussian09 with B3LYP (functional).

It will be understood that modelling data is not measured in the same way as SWV as described herein.

HOMO and LUMO levels for acceptor (ACC) of model compounds of General Formula 1 were modelled:

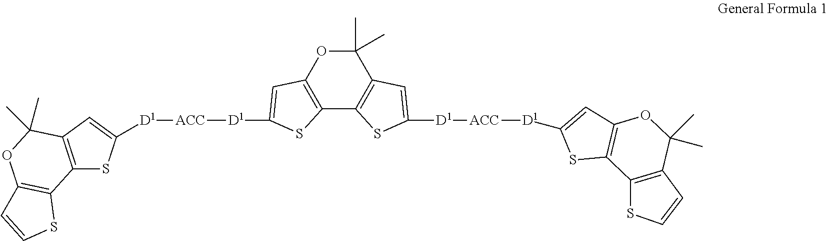

TABLE 1

| | | HOMO | LUMO | E_{g} | Abs |
|---|---|---|---|---|---|
| Entry | D¹-ACC-D¹ | (eV) | (eV) | (eV) | (nm) |
| Comparative Example 1-1 | 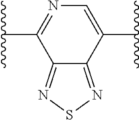 [SMILES: *c1cnc(*)c2nsnc12] | −4.60 | −3.06 | 1.54 | 803 |
| Model Example 1-1 | 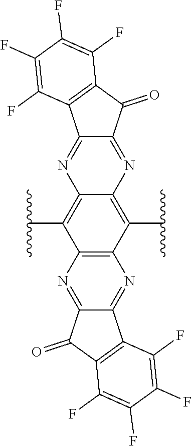 [SMILES: *c1c2nc3c(=O)c4c(F)c(F)c(F)c(F)c4c3nc2c(*)c2nc3c(=O)c4c(F)c(F)c(F)c(F)c4c3nc12] | −4.59 | −3.72 | 0.87 | 1430 |
| Model Example 1-2 | 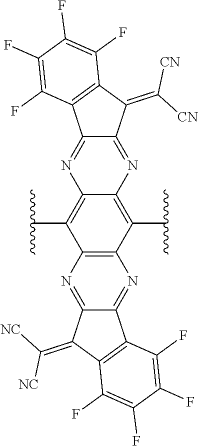 [SMILES: *c1c2nc3c(nc2c(*)c2nc4c(nc12)-c1c(F)c(F)c(F)c(F)c1C4=C(C#N)C#N)-c1c(F)c(F)c(F)c(F)c1C3=C(C#N)C#N] | −4.82 | −4.10 | 0.72 | 1724 |
| Model Example 1-3 | 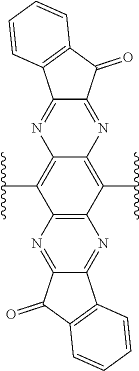 [SMILES: *c1c2nc3c(=O)c4ccccc4c3nc2c(*)c2nc3c(=O)c4ccccc4c3nc12] | −4.44 | −3.40 | 1.04 | 1190 |
| Model Example 1-4 | 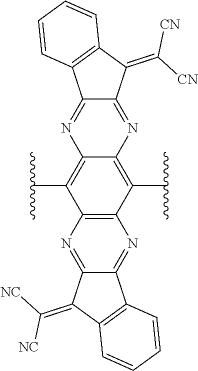 [SMILES: *c1c2nc3c(nc2c(*)c2nc4c(nc12)-c1ccccc1C4=C(C#N)C#N)-c1ccccc1C3=C(C#N)C#N] | −4.65 | −3.88 | 0.77 | 1608 |
| Model Example 1-5 | 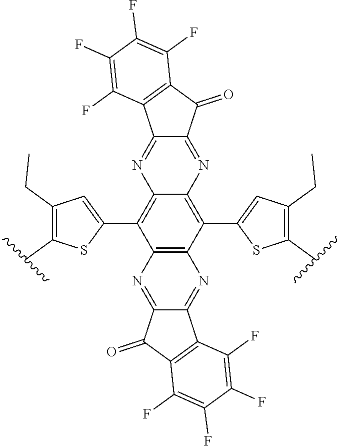 [SMILES: *c1sc(-c2c3nc4c(=O)c5c(F)c(F)c(F)c(F)c5c4nc3c(-c3cc(CC)c(*)s3)c3nc4c(=O)c5c(F)c(F)c(F)c(F)c5c4nc23)cc1CC] | −4.51 | −3.58 | 0.93 | 1340 |
| Model Example 1-6 | 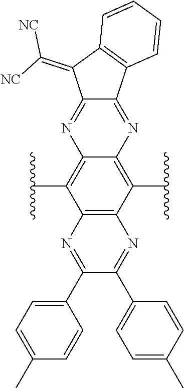 [SMILES: *c1c2nc3c(nc2c(*)c2nc(-c4ccc(C)cc4)c(-c4ccc(C)cc4)nc12)-c1ccccc1C3=C(C#N)C#N] | −4.47 | −3.43 | 1.04 | 1194 |
| Model Example 1-7 | 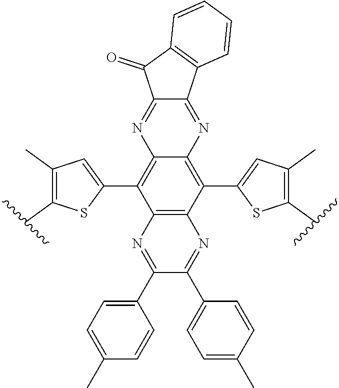 [SMILES: *c1sc(-c2c3nc4c(nc3c(-c3cc(C)c(*)s3)c3nc(-c5ccc(C)cc5)c(-c5ccc(C)cc5)nc23)-c2ccccc2C4=O)cc1C] | −4.28 | −3.10 | 1.18 | 1051 |
| Model Example 1-8 | 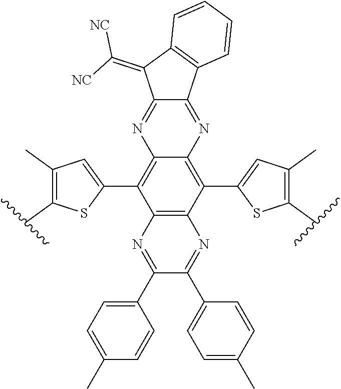 [SMILES: *c1sc(-c2c3nc4c(nc3c(-c3cc(C)c(*)s3)c3nc(-c5ccc(C)cc5)c(-c5ccc(C)cc5)nc23)-c2ccccc2C4=C(C#N)C#N)cc1C] | −4.41 | −3.38 | 1.03 | 1201 |
| Model Example 1-9 | 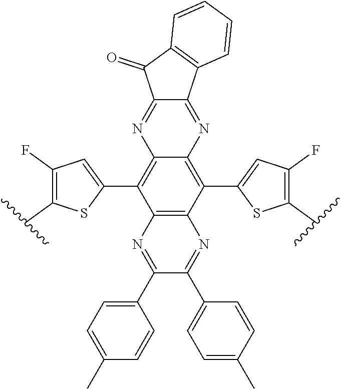 [SMILES: *c1sc(-c2c3nc4c(nc3c(-c3cc(F)c(*)s3)c3nc(-c5ccc(C)cc5)c(-c5ccc(C)cc5)nc23)-c2ccccc2C4=O)cc1F] | −4.39 | −3.27 | 1.12 | 1107 |
| Model Example 1-10 | 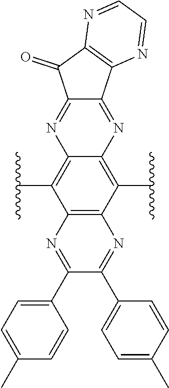 [SMILES: *c1c2nc3c(nc2c(*)c2nc(-c4ccc(C)cc4)c(-c4ccc(C)cc4)nc12)-c1nccnc1C3=O] | −4.39 | −3.33 | 1.06 | 1172 |
| Model Example 1-11 | 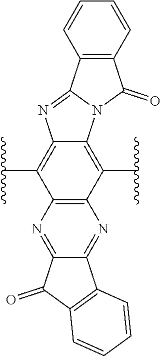 [SMILES: *c1c2nc3c(=O)c4ccccc4c3nc2c(*)c2c1nc1c3ccccc3c(=O)n12] | −4.48 | −2.87 | 1.61 | 770 |
| Model Example 1-12 | 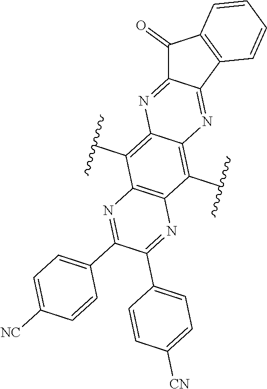 [SMILES: *c1c2nc3c(nc2c(*)c2nc(-c4ccc(C#N)cc4)c(-c4ccc(C#N)cc4)nc12)-c1ccccc1C3=O] | −4.65 | −3.50 | 1.16 | 1073 |
| Model Example 1-13 | 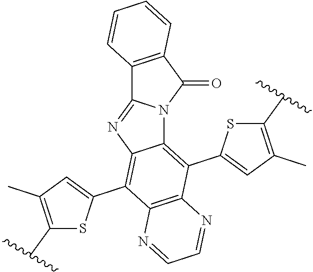 | −4.37 | −2.75 | 1.62 | 767 |
| Model Example 1-14 | 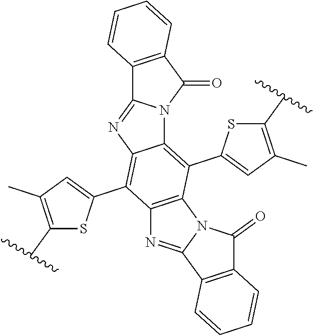 | −4.50 | −2.43 | 2.06 | 601 |
| Model Example 1-15 | 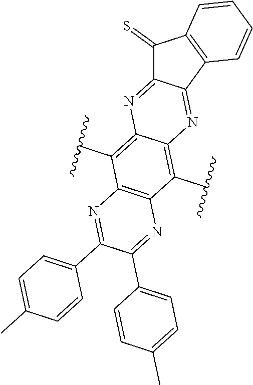 [SMILES: *c1c2nc3c(nc2c(*)c2nc(-c4ccc(C)cc4)c(-c4ccc(C)cc4)nc12)-c1ccccc1C3=S] | −4.35 | −3.34 | 1.01 | 1223 |
| Model Example 1-16 | 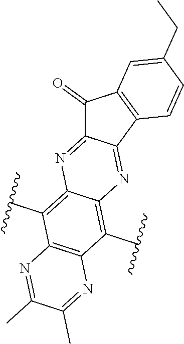 [SMILES: *c1c2nc(C)c(C)nc2c(*)c2nc3c(nc12)C(=O)c1cc(CC)ccc1-3] | −4.29 | −3.08 | 1.211 | 1024 |

### Quantum Chemical Modelling Example 2

HOMO and LUMO levels for acceptor (ACC) of model compounds of General Formula 2 were modelled:

TABLE 2

| | | | HOMO | LUMO | E_{g} | Abs |
|---|---|---|---|---|---|---|
| Name | D | D1-ACC-D1 | (eV) | (eV) | (eV) | (nm) |
| Comparative Example 2-1 | 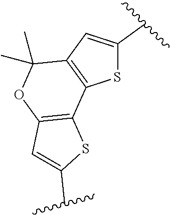 [SMILES: *c1cc2c(s1)-c1sc(*)cc1C(C)(C)O2] | 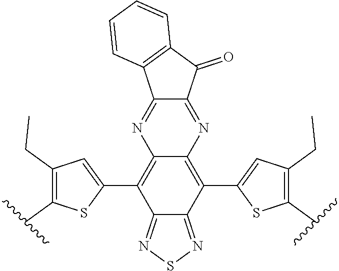 [SMILES: *c1sc(-c2c3nsnc3c(-c3cc(CC)c(*)s3)c3nc4c(nc23)C(=O)c2ccccc2-4)cc1CC] | −4.45 | −3.35 | 1.10 | 1128 |
| Comparative Example 2-2 | 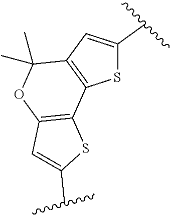 [SMILES: *c1cc2c(s1)-c1sc(*)cc1C(C)(C)O2] | 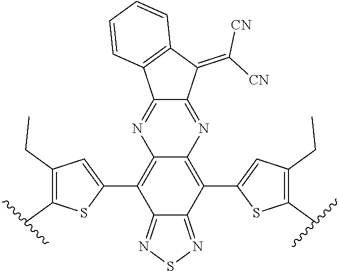 [SMILES: *c1sc(-c2c3nsnc3c(-c3cc(CC)c(*)s3)c3nc4c(nc23)C(=C(C#N)C#N)c2ccccc2-4)cc1CC] | −4.60 | −3.60 | 1.00 | 1237 |
| Comparative Example 2-3 | 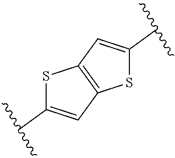 [SMILES: *c1cc2sc(*)cc2s1] | 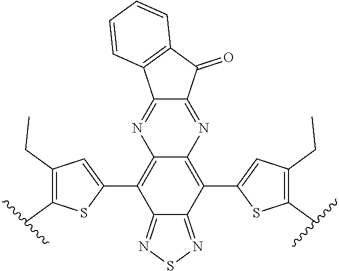 [SMILES: *c1sc(-c2c3nsnc3c(-c3cc(CC)c(*)s3)c3nc4c(nc23)C(=O)c2ccccc2-4)cc1CC] | −4.62 | −3.46 | 1.16 | 1065 |
| Comparative Example 2-4 | 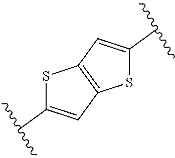 [SMILES: *c1cc2sc(*)cc2s1] | 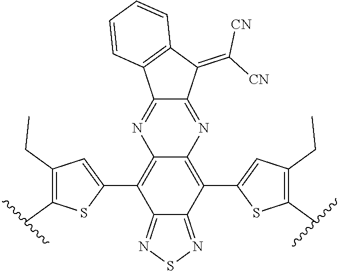 [SMILES: *c1sc(-c2c3nsnc3c(-c3cc(CC)c(*)s3)c3nc4c(nc23)C(=C(C#N)C#N)c2ccccc2-4)cc1CC] | −4.80 | −3.71 | 1.09 | 1140 |

### D-D¹ACC-D¹-D-D¹-ACC-D¹-D

### General Formula 2

### Device Example 1

A device having the following structure was prepared:
Cathode/Donor:Acceptor layer/Anode

A glass substrate coated with a layer of indium-tin oxide (ITO) was treated with polyethyleneimine (PETE) to modify the work function of the ITO.

A mixture of Polymer Example 1 (donor) and ITIC-4F (acceptor) in a donor:acceptor mas ratio of 1:1.5 was deposited over the modified ITO layer by blade coating from a 15 mg/ml solution in 1,2,4 Trimethylbenzene; 1,2-Dimethoxybenzene 95:5 v/v solvent mixture. The film was dried at 80° C. to form a ca. 500 nm thick bulk heterojunction layer

An anode stack of MoO₃ (10 nm) and ITO (50 nm) was formed over the bulk heterojunction by thermal evaporation (Moos) and sputtering (ITO)

Device Example 2 was prepared as described for Device Example 1 except that Polymer Example 2 was used in place of Polymer Example 1.

### Comparative Device 1

A comparative device was prepared as described for Device Example 1 except that Comparative Polymer 1 was used in place of Polymer Example 1 and the thickness of the ITO was 150 nm.

Comparative Devices 2 and 3 were prepared as described for Device Example 1 except that Comparative Polymer 2 and 3 respectively, were used in place of Polymer Example 1.

In Comparative Polymer 1, R⁵⁴ is 3,7-dimethyloctyl for 50% of n and is C12H25 for the other 50%.

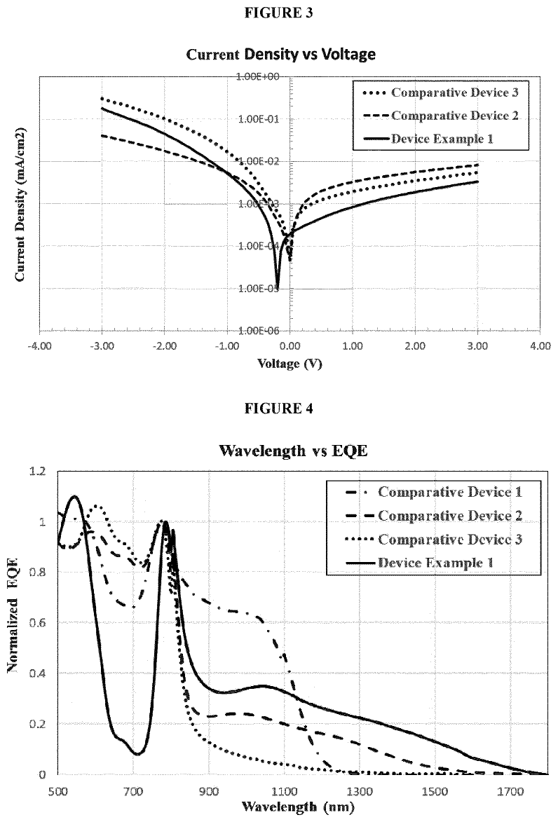

With reference to FIG. 3, Device Example 1 has a lower dark current than either Comparative Device 2 or 3.

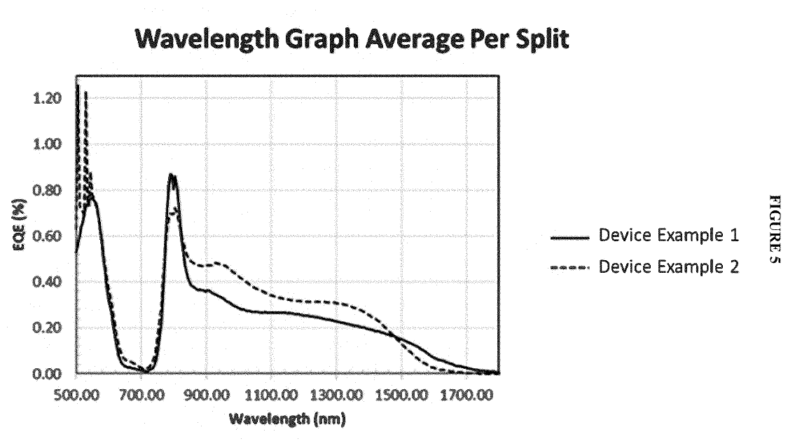

With reference to FIG. 4, Device Example 1 has higher efficiency than any of the comparative devices at wavelengths about 1200 nm.

With reference to FIG. 5, Device Example 2 has higher efficiency than Device Example 1 in the range of about 900-1400 nm.

1. A material comprising an electron-accepting unit of formula (I): 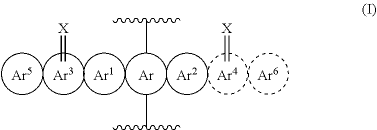
Ar is an aromatic ring;
Ar¹ is a substituted or unsubstituted 5- or 6-membered heteroaromatic ring containing N and C ring atoms;
when Ar¹ is a substituted or unsubstituted 6-membered heteroaromatic ring, Ar² is a substituted or unsubstituted 6-membered heteroaromatic ring wherein the ring atoms are selected from N and C;
when Ar¹ is a 5-membered heteroaromatic ring, Ar² is a substituted or unsubstituted 5- or 6-membered heteroaromatic ring;
Ar³ is a 5-membered ring or a substituted or unsubstituted 6-membered ring;
Ar⁴ is a 5-membered ring or a substituted or unsubstituted 6-membered ring or is absent;
Ar⁵ is a substituted or unsubstituted monocyclic or polycyclic group containing at least one aromatic or heteroaromatic ring;
Ar⁶ is a substituted or unsubstituted monocyclic or polycyclic group containing at least one aromatic or heteroaromatic ring or is absent; and each
X is independently a substituent bound to a C atom of Ar³, and where present Ar⁴, with the proviso that at least one X is an electron withdrawing group;
and wherein the material further comprises an electron-donating unit D.

2. The material according to claim 1 wherein the material is selected from formula (I-1) to (I-21): 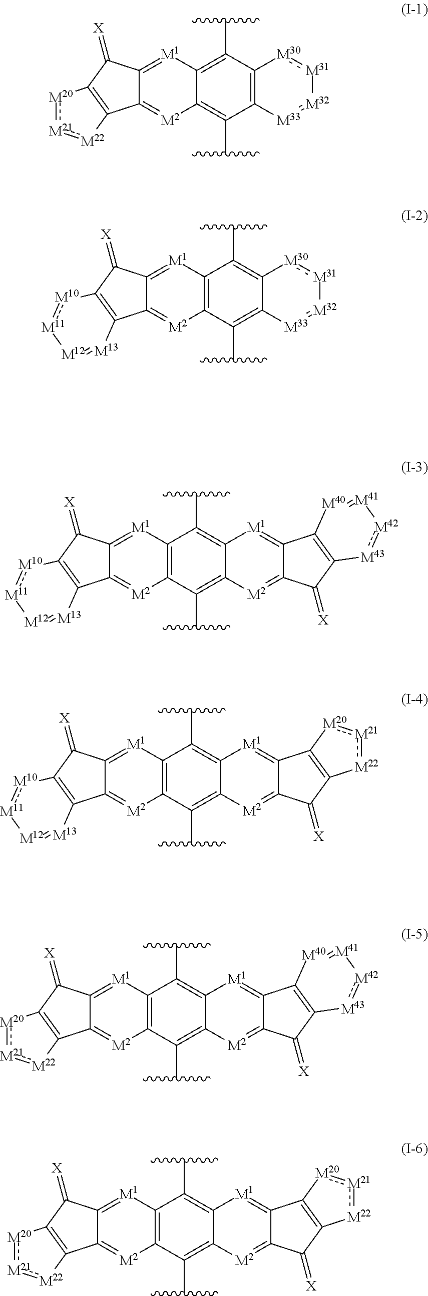 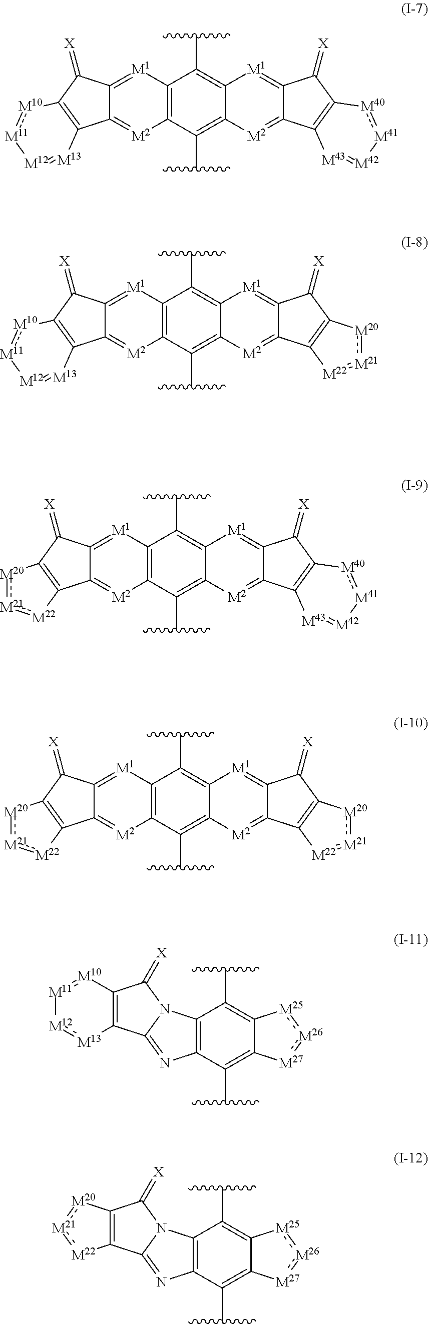 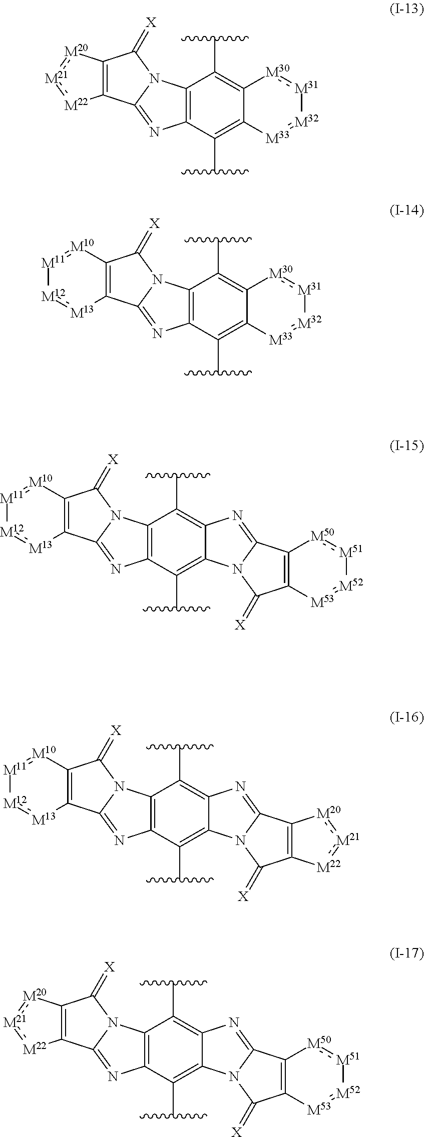 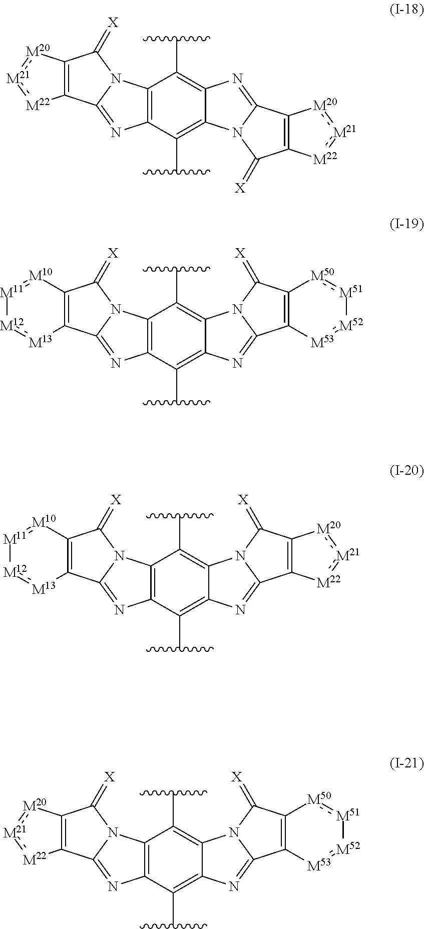
wherein
M¹ and M² is independently CR⁶¹ or N wherein R⁶¹ in each occurrence is H or a substituent;
M¹⁰, m¹¹, m¹², m¹³, M²⁰, M²¹, M²², M⁴⁰, M⁴¹, M⁴², M⁴³, M⁵⁰, M⁵¹, M⁵², and M⁵³ is independently N, S, O or CR⁶¹, with the proviso that a S or O is not adjacent to another S or O;
M³⁰, m³¹, M³² and M³³ is independently N, or CR⁶¹;
m²⁵, M²⁶ and M²⁷ is independently N, S, O or CR⁶¹; and with the proviso that a N or O is not adjacent to another N or O; and
X is independently an electron withdrawing group.

3. A material according to claim 1 wherein each electron-withdrawing group X is independently selected from O, S and NX⁷⁰ wherein X⁷⁰ is selected from CN; COOR⁸⁰; C₁ to C₂₀ alkyl where one or more non-adjacent, not terminal C may be replaced by O or S; and a substituted or unsubstituted 5- or 6-membered aromatic or heteroaromatic ring; and CX¹⁰X¹¹ wherein X¹⁰ and X¹¹ is each independently F, CN, CF₃ or COOR⁸⁰, wherein R⁸⁰ is H or a substituent.

4. The material according to claim 1 wherein each X is an electron-withdrawing group.

5. The material according to claim 1 wherein the material is a non-polymeric compound.

6. The material according to claim 5 wherein the material is selected form formula (Ia) or (Ib): 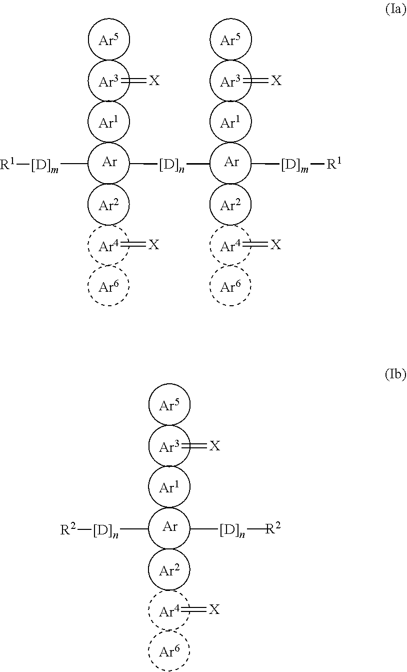
wherein n is at least 1; m is 0, 1, 2 or 3; D in each occurrence is independently an electron-donating unit which may be unsubstituted or substituted with one or more substituents; and
R¹ and R² independently in each occurrence is H or a substituent.

7. The material according to claim 1 wherein the material is a polymer; the unit of formula (I) is an electron-accepting repeat unit of formula (I); and the electron-donating unit D is an electron-donating repeat unit.

8. The material according to claim 1 wherein D is selected from formulae (IIa)-(IIq): 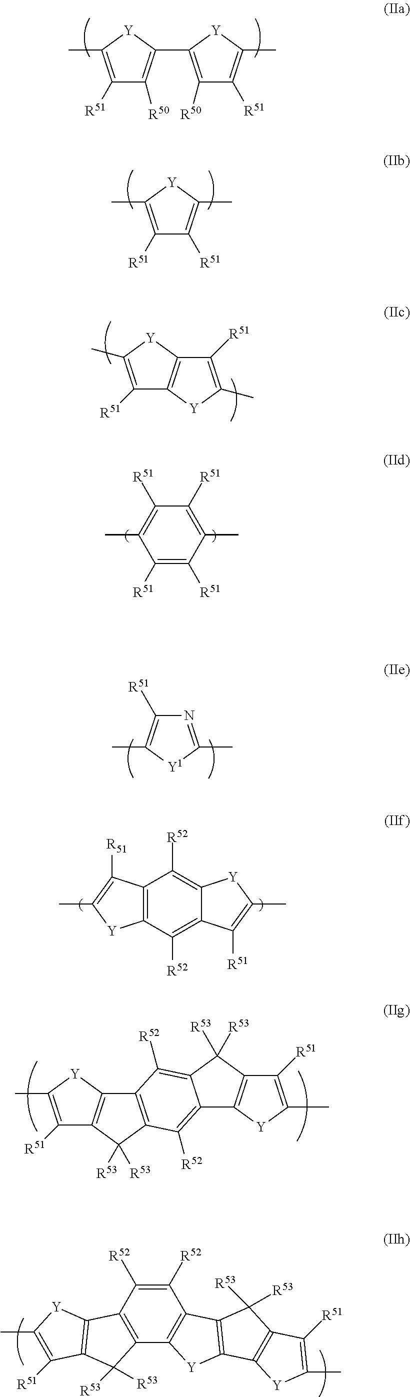 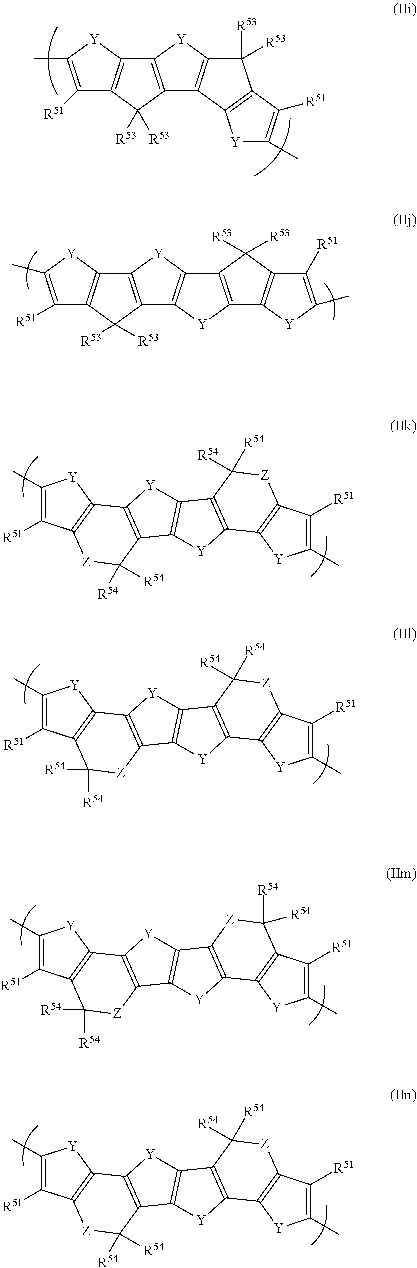 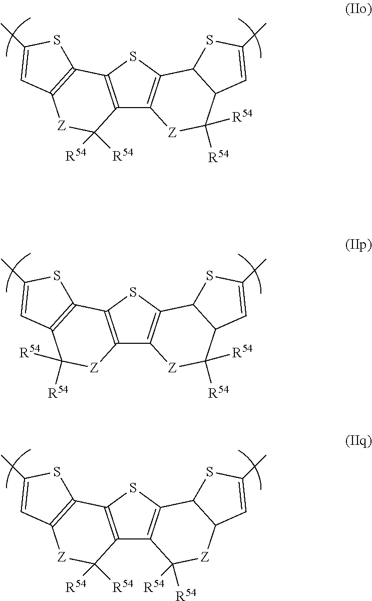
wherein Y and Y¹ in each occurrence is independently O or S, Z in each occurrence is O, S, NR⁵⁵ or C(R⁵⁴)₂; R⁵⁰, R⁵¹, R⁵² R⁵⁴ and R⁵⁵ independently in each occurrence is H or a substituent wherein R⁵⁰ groups may be linked to form a ring; and R⁵³ independently in each occurrence is a substituent.

9. A polymer comprising a repeat unit of formula (I): 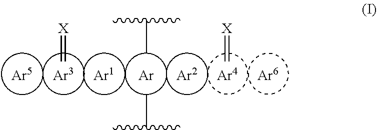
wherein:
Ar is an aromatic ring;
Ar l is a substituted or unsubstituted 5- or 6-membered heteroaromatic ring containing N and C ring atoms;
when Ar¹is a substituted or unsubstituted 6-membered heteroaromatic ring, Ar² is a substituted or unsubstituted 6-membered heteroaromatic ring wherein the ring atoms are selected from N and C;
when Ar¹ is a 5-membered heteroaromatic ring, Ar² is a substituted or unsubstituted 5- or 6-membered heteroaromatic ring;
Ar³ is a 5-membered ring or a substituted or unsubstituted 6-membered ring;
Ar⁴ is a 5-membered ring or a substituted or unsubstituted 6-membered ring or is absent;
Ar⁵ is a substituted or unsubstituted monocyclic or polycyclic group containing at least one aromatic or heteroaromatic ring;
Ar⁶ is a substituted or unsubstituted monocyclic or polycyclic group containing at least one aromatic or heteroaromatic ring or is absent; and each X is independently a substituent bound to a C atom of Ar³, and where present, Ar⁴ with the proviso that at least one X is an electron withdrawing group.

10. A polymer according to claim 9 where the repeat unit of formula (I) is selected from formulae (I-1) to (I-21):

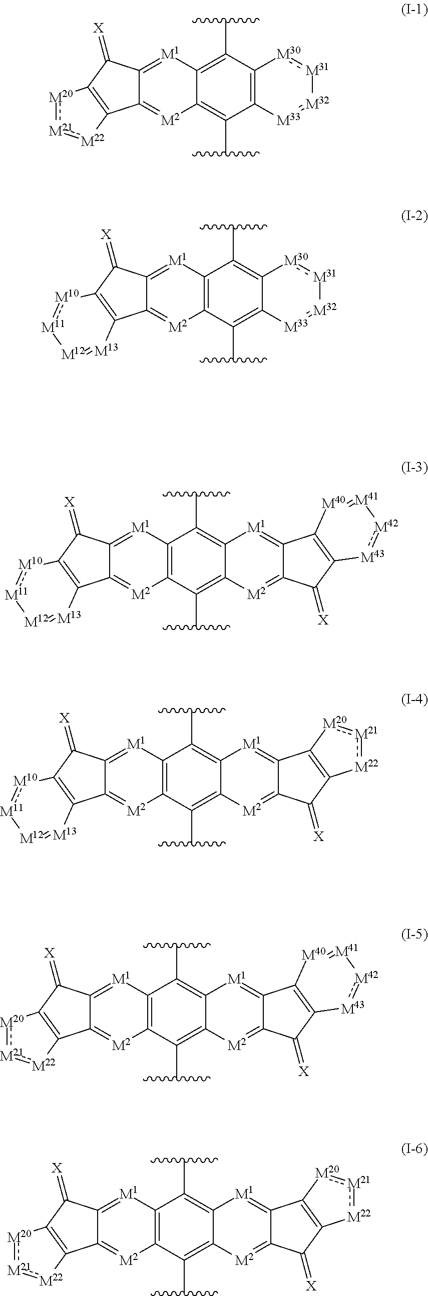

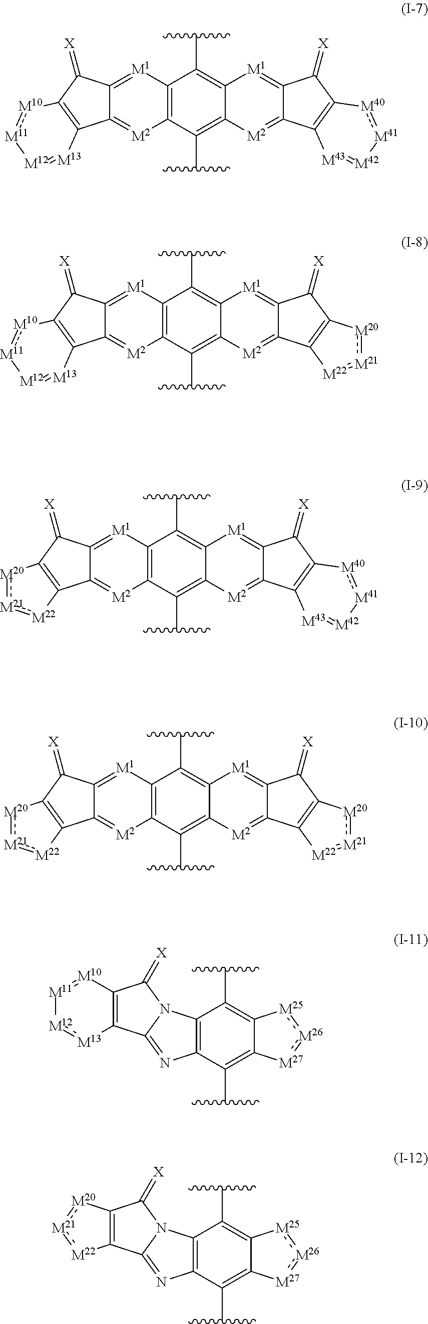

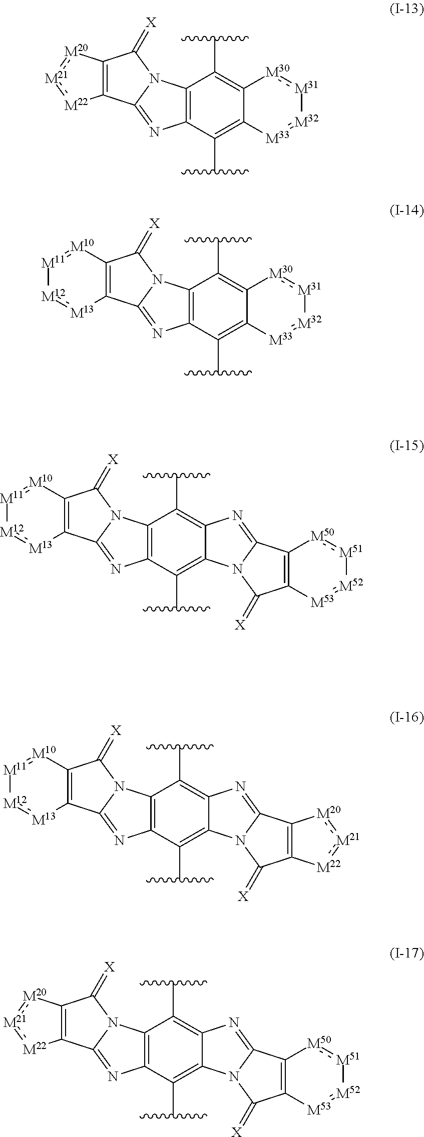

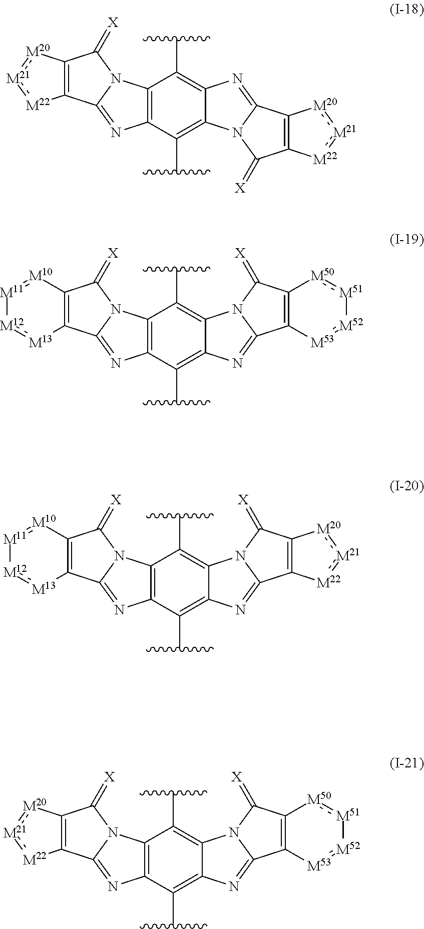

11. A polymer according to claim 9 wherein the polymer comprises an electron-donating repeat unit D.

12. A polymer according to claim 11 wherein the electron-donating repeat unit D comprises a fused or unfused furan or thiophene.

13. A polymer of according to claim 11 wherein the electron-donating repeat unit D is selected from formulae (IIa)-(IIq) and combinations thereof: 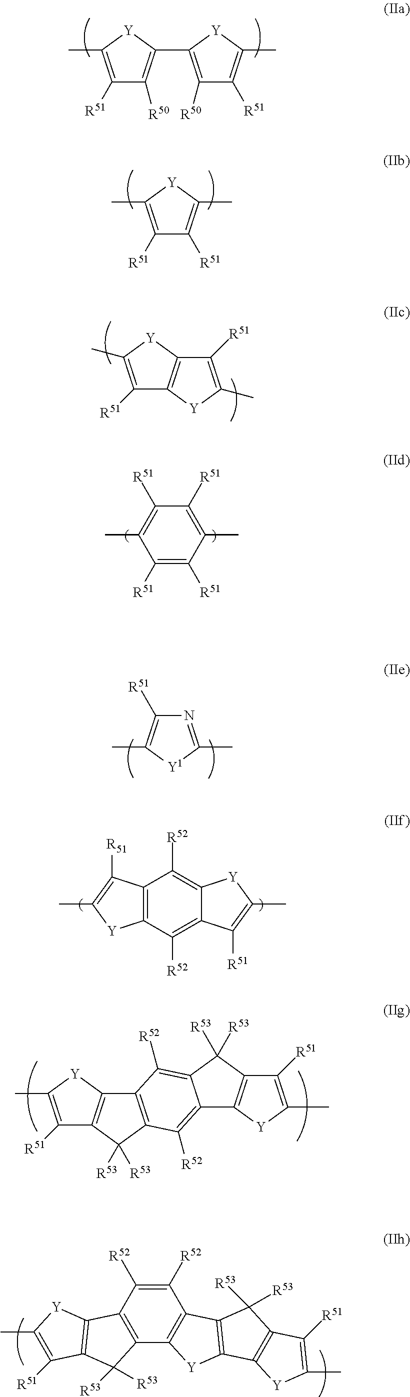 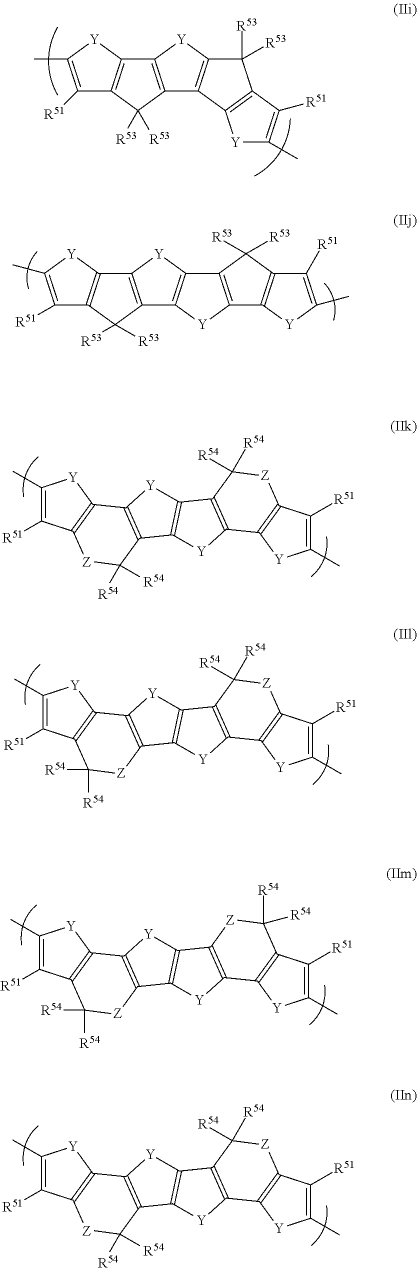 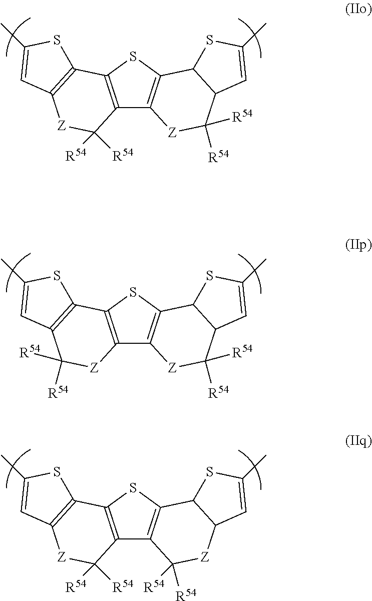
wherein Y in each occurrence is independently O or S, Z in each occurrence is O, S, NR⁵⁵ or C(R⁵⁴)₂; R⁵⁰, R⁵¹, R⁵² R⁵⁴ and R⁵⁵ independently in each occurrence is H or a substituent wherein R⁵⁰ groups may be linked to form a ring; and R⁵³ independently in each occurrence is a substituent.

14. A composition comprising an electron donor and an electron acceptor wherein at least one of the electron donor and electron acceptor is a material or polymer according to claim 1.

15. The composition according to claim 14 wherein the electron acceptor is the material comprising an electron-accepting unit of formula (I).

16. The composition according to claim 15 wherein the electron acceptor is a non-polymeric compound.

17. The composition comprising an electron donor and an electron acceptor wherein at least one of the electron donor and electron acceptor is a material or polymer comprising an electron-accepting unit of formula (I) wherein the electron donor is the polymer according to claim 9.

18. An organic electronic device comprising an active layer comprising a material or composition according to claim 1.

19. An organic electronic device according to claim 18 wherein the organic electronic device is an organic photoresponsive device comprising a bulk heterojunction layer disposed between an anode and a cathode and wherein the bulk heterojunction layer comprises a composition comprising an electron donor and an electron acceptor wherein at least one of the electron donor and electron acceptor is a material or polymer comprising an electron-accepting unit of formula (I).

20. An organic electronic device according to claim 19 wherein the organic photoresponsive device is an organic photodetector.

21. A photosensor comprising a light source and an organic photodetector according to claim 20, wherein the photosensor is configured to detect light emitted from a light source.

22. A photosensor according to claim 21, wherein the light source emits light having a peak wavelength of at least >1200 nm.

23. A formulation comprising a material, polymer or composition according to claim 1 dissolved or dispersed in one or more solvents.

24. A method of forming an organic electronic device according to claim 18 wherein formation of the active layer comprises deposition of a formulation comprising a material, polymer or composition comprising an electron-accepting unit of formula (I) dissolved or dispersed in one or more solvents onto a surface and evaporation of the one or more solvents.
